SEQUENCE LISTING (29 sequences)

SEQ ID NO: 1 - DNA, 15 bp, Mus musculus
tcaagtgtaacttac

SEQ ID NO: 2 - DNA, 15 bp, Mus musculus
tcaagtgtaagttac

SEQ ID NO: 3 - DNA, 15 bp, Mus musculus
tcaagtataacttac

SEQ ID NO: 4 - DNA, 9 bp, Mus musculus
gacacatcc

SEQ ID NO: 5 - DNA, 27 bp, Mus musculus
cagcagtacagtggtcacccatcctcg

SEQ ID NO: 6 - DNA, 27 bp, Mus musculus
caacagtacagtggttacccgtacacg

SEQ ID NO: 7 - DNA, 27 bp, Mus musculus
cagcagtacagtggttacccatccacg

SEQ ID NO: 8 - DNA, 24 bp, Mus musculus
ggcttcaacatcaaagacacctat

SEQ ID NO: 9 - DNA, 24 bp, Mus musculus
ggcttcaacattaaagacacctat

SEQ ID NO: 10 - DNA, 24 bp, Mus musculus
attgatcctgcgattgataatact

SEQ ID NO: 11 - DNA, 24 bp, Mus musculus
attgatcctgcgattggtaatact

SEQ ID NO: 12 - DNA, 24 bp, Mus musculus
attgatcctgcgattggtaatcct

SEQ ID NO: 13 - DNA, 27 bp, Mus musculus
gcttctgggttctatactatggactac

SEQ ID NO: 14 - DNA, 27 bp, Mus musculus
gctagagggttctatggtatggactac

SEQ ID NO: 15 - DNA, 27 bp, Mus musculus
gcttctgggttctatgctatggactgc

SEQ ID NO: 16 - DNA, 318 bp, Mus musculus
gcaaattgttctcacccagtctccagcaatcatgtctgcatctccaggggagaaggtcaccatgacctgcagtgccagctcaagtgtaacttacatgtattggttccagcagaagccaggctcctcccccagactctggatttatgacacatccaacctggtttctggagtccctgctcgcttcagtggcagtaggtctgggacctcttattctctcacaatcagcatatggaggctgaagatgctgccacttattactgccagcagtacagtggtcacccatcctcgttcggctcggggacaaagttggaaattaaa

SEQ ID NO: 17 - DNA, 318 bp, Mus musculus
caaattgttctcacccagtctccagcaatcatgtctgcatctccaggggagaaggtcaccatgacctgcagtgccagctcaagtgtaagttacatgttctggtaccagcagaagccaggctcctcccccagactctggatttatgacacatccaacctggtttctggagtccctgctcgcttcagtggcagtaggtctgggacctcttattctctcacaatcagcagcatggaggctgaagatgctgccacttattactgccaacagtacagtggttacccgtacacgttcggaggggggaccaagctggaaataaag

SEQ ID NO: 18 - DNA, 318 bp, Mus musculus
caaattgttctcacccagtctccagccatcatgtctgcatctccaggggaaaaggtcaccatgacctgcagtgccagctcaagtataacttacatgttctggtaccagcagaagccaggctcctcccccagactctggatttatgacacatccaacctggtttctggagtccctgctcgcttcagtggcagtaagtctgggacctcttattctctcacaatcaccagcatggaggctgaagatgctgccacttattactgccagcagtacagtggttacccatccacgttcggctcggggacaaagttggaaataaaa

SEQ ID NO: 19 - DNA, 348 bp, Mus musculus
gaggttcagctgcagcagtctggggcagagcttgtgaagccaggggcctcagtcaagttgtcctgcacagcttctggcttcaacatcaaagacacctatatgcattgggtgaagcagaggcctgaacagggcctggagtggattggaaggattgatcctgcgattgataatactaaatatgacccgaagttccagggcaaggccactataacagctgacacatcctccaacacagcctacctgcagctcagcagcctgacatctgaggacactgccgtctattactgtgcttctgggttctatactatggactactggggtcaaggaacctcagtcaccgtctcctca

SEQ ID NO: 20 - DNA, 348 bp, Mus musculus
gaggttcggctgcagcagtctggggcagagcttgtgaagccaggggcctcagtcaagttgtcctgcacagcttctggcttcaacattaaagacacctatttacactggttgaaggagaggcctgaacagggcctggagtggattggaaggattgatcctgcgattggtaatactagatatgacccgaagttccaggtcaaggccactataacagcagacacatcctccaacacagcctacctgcaactcagcagcctgacatctgaggactctgccgtctattactgtgctagagggttctatggtatggactactggggtcaaggaacctcagtcaccgtctcctca

SEQ ID NO: 21 - DNA, 348 bp, Mus musculus
gaggttcagctgcagcagtctggggcagaggttgtgaagccaggggcctcagtcaagttgtcctgcacagcttctggcttcaacattaaagacacctatatgcactgggtgaagcagaggcctgaacagggcctggagtggattggaaggattgatcctgcgattggtaatcctaaatatgacccgaagttccagggcagggccactataactgctgacacatcctccaacacagcctacctgcagctcagcagcctgacatctgaggacactgccgtctattactgtgcttctgggttctatgctatggactgctggggtcaaggaacctcagtcaccgtctcctca

SEQ ID NO: 22 - protein, 106 aa, Mus musculus
QIVLTQSPAIMSASPGEKVTMTCSASSSVTYMYWFQQKPGSSPRLWIYDTSNLVSGVPARFSGSRSGTSYSLTISSMEAEDAATYYCQQYSGHPSSFGSGTKLEIK

SEQ ID NO: 23 - protein, 106 aa, Mus musculus
QIVLTQSPAIMSASPGEKVTMTCSASSSVSYMFWYQQKPGSSPRLWIYDTSNLVSGVPARFSGSRSGTSYSLTISSMEAEDAATYYCQQYSGYPYTFGGGTKLEIK

SEQ ID NO: 24 - protein, 106 aa, Mus musculus
QIVLTQSPAIMSASPGEKVTMTCSASSSITYMFWYQQKPGSSPRLWIYDTSNLVSGVPARFSGSKSGTSYSLTITSMEAEDAATYYCQQYSGYPSTFGSGTKLEIK

SEQ ID NO: 25 - protein, 116 aa, Mus musculus
EVQLQQSGAELVKPGASVKLSCTASGFNIKDTYMHWVKQRPEQGLEWIGRIDPAIDNTKYDPKFQGKATITADTSSNTAYLQLSSLTSEDTAVYYCASGFYTMDYWGQGTSVTVSS

SEQ ID NO: 26 - protein, 116 aa, Mus musculus
EVRLQQSGAELVKPGASVKLSCTASGFNIKDTYLHWLKERPEQGLEWIGRIDPAIGNTRYDPKFQVKATITADTSSNTAYLQLSSLTSEDSAVYYCARGFYGMDYWGQGTSVTVSS

SEQ ID NO: 27 - protein, 116 aa, Mus musculus
EVQLQQSGAEVVKPGASVKLSCTASGFNIKDTYMHWVKQRPEQGLEWIGRIDPAIGNPKYDPKFQGRATITADTSSNTAYLQLSSLTSEDTAVYYCASGFYAMDCWGQGTSVTVSS

SEQ ID NO: 28 - protein, 1029 aa, Canis lupus
TGGGGTCAAGGAACCTCAGTCACCGTCTCCTCAGCTAGCACCACGGCCCCCTCGGTTTTCCCACTGGCCCCCAGCTGCGGGTCCACTTCCGGCTCCACGGTGGCCCTGGCCTGCCTGGTGTCAGGCTACTTCCCCGAGCCTGTAACTGTGTCCTGGAATTCCGGCTCCTTGACCAGCGGTGTGCACACCTTCCCGTCCGTCCTGCAGTCCTCAGGGCTTCACTCCCTCAGCAGCATGGTGACAGTGCCCTCCAGCAGGTGGCCCAGCGAGACCTTCACCTGCAACGTGGTCCACCCAGCCAGCAACACTAAAGTAGACAAGCCAGTGTTCAATGAATGCAGATGCACTGATACACCCCCATGCCCAGTCCCTGAACCTCTGGGAGGGCCTTCGGTCCTCATCTTTCCCCCGAAACCCAAGGACATCCTCAGGATTACCCGAACACCCGAGGTCACCTGTGTGGTGTTAGATCTGGGCCGTGAGGACCCTGAGGTGCAGATCAGCTGGTTCGTGGATGGTAAGGAGGTGCACACAGCCAAGACCCAGTCTCGTGAGCAGCAGTTCAACGGCACCTACCGTGTGGTCAGCGTCCTCCCCATTGAGCACCAGGACTGGCTCACAGGGAAGGAGTTCAAGTGCAGAGTCAACCACATAGACCTCCCGTCTCCCATCGAGAGGACCATCTCTAAGGCCAGAGGGAGGGCCCATAAGCCCAGTGTGTATGTCCTGCCGCCATCCCCAAAGGAGTTGTCATCCAGTGACACAGTCAGCATCACCTGCCTGATAAAAGACTTCTACCCACCTGACATTGATGTGGAGTGGCAGAGCAATGGACAGCAGGAGCCCGAGAGGAAGCACCGCATGACCCCGCCCCAGCTGGACGAGGACGGGTCCTACTTCCTGTACAGCAAGCTCTCTGTGGACAAGAGCCGCTGGCAGCAGGGAGACCCCTTCACATGTGCGGTGATGCATGAAACTCTACAGAACCACTACACAGATCTATCCCTCTCCCATTCTCCGGGTAAATGA

SEQ ID NO: 29 - protein, 333 aa, Canis lupus
CGAAATGATGCCCAGCCAGCCGTCTATTTGTTCCAACCATCTCCAGACCAGTTACACACAGGAAGTGCCTCTGTTGTGTGCTTGCTGAATAGCTTCTACCCCAAAGACATCAATGTCAAGTGGAAAGTGGATGGTGTCATCCAAGACACAGGCATCCAGGAAAGTGTCACAGAGCAGGACAAGGACAGTACCTACAGCCTCAGCAGCACCCTGACGATGTCCAGTACTGAGTACCTAAGTCATGAGTTGTACTCCTGTGAGATCACTCACAAGAGCCTGCCCTCCACCCTCATCAAGAGCTTCCAAAGGAGCGAGTGTCAGAGAGTGGACTAG

(19) United States
(12) Patent Application Publication
(10) Pub. No.: US 2022/0324977 A1
(43) Pub. Date: Oct. 13, 2022

(54) ANTI-CANINE PD-1 ANTIBODY BINDING WITH CANINE PD-1

(71) Applicant: BEIJING SINOGENE BIOTECHNOLOGY CO., LTD., Beijing (CN)

(72) Inventors: Jidong MI, Beijing (CN); Jianping ZHAO, Beijing (CN); Min ZHENG, Beijing (CN); Fan HUANG, Sunnyvale, CA (US)

(21) Appl. No.: 17/616,103

(22) PCT Filed: Jun. 3, 2020
(86) PCT No.: PCT/CN2020/094243

§ 371 Date: Dec. 2, 2021

(30) Foreign Application Priority Data
Jun. 3, 2019 (CN) 201910480349.2

(51) Int. Cl. C07K 16/28; A61P 35/00

(52) U.S. Cl. CPC C07K 16/2818; A61P 35/00; A61K 2039/505

(57) ABSTRACT

Provided is an anti-canine PD-1 antibody, capable of binding with canine PD-1 and comprising three light-chain complementary determining regions (CDR1-3) or a conservatively modified variant maintaining its function and/or three heavy-chain complementary determining regions (CDR1-3) or a conservatively modified variant maintaining its function. Further provided is an application of the anti-canine PD-1 antibody in the preparation of medicines for treating canine cancers.

### TECHNICAL FIELD

The present disclosure relates to an anti-canine PD-1 antibody binding with canine PD-1 and a preparation method thereof.

### BACKGROUND

At present, there are more than 400 canine varieties acknowledged throughout the world. The genetic variation level of the canine varieties may be as high as humans. With prolonged lifetime of the canines, the cancer morbidity in canines has gradually increased over the past 20 years. More than 1.66 million people (about 500/100,000) and more than 4.2 million canines (about 5300/100,000) are diagnosed with cancers each year in USA. Some purebred dogs are more susceptible to a specific cancer, and sometimes even susceptible to multiple types of cancers. Compared with human patients, early diagnosis of a cancer in canine is challenging, and in general, it is definitively diagnosed at an advanced stage of the cancer. Cancers that are common to both canine and human include sarcoma (osteosarcoma, soft tissue sarcoma, histiocytosarcoma and hemangiosarcoma), hematological malignant tumors (lymphoma and leukemia), bladder cancer, intracranial tumors (meningeoma and glioma) and melanoma.

Programmed cell death protein 1 (PD-1) is an important immunosuppressive molecule. Immunoregulation targeting PD-1 is of great significance for anti-tumor, anti-infection, anti-autoimmune disease, organ graft survival and the like. PD-1 is an inhibitory T cell receptor which mainly works in tumor microenvironment through two known ligands, PD-L1 (also called B7-H1 or CD274) and PD-L2 (also called B7-DC or CD273).

Moreover, PD-L1 expressed on the surface of antigen-presenting cells can bind to CD80 on T cells, thereby inhibiting immune response. In the micro-environment of tumor, the activated PD-1/PD-L1 signaling pathway can reduce tumor-specific T cell immune effect, thereby mediating tumor immune escape and accelerating tumor growth.

Duraiswamy, et al. found that the depletion of tumor infiltrating lymphocytes (TILs) in tumor microenvironment was closely related to PD-L1 expressed by tumor cells and myeloid-derived suppressor cells (tumor-associated macrophages, dendritic cells and the like). The blocking of PD-1/PD-L1 signaling pathway can enhance the functions of effector CD8 T cells, and inhibit the functions of the Treg cells and myeloid-derived suppressor cells, thereby enhancing the anti-tumor effect of immune system.

Thus, the canine cancers can be good spontaneous cancer comparison models, and the therapeutic methods for the canine cancers usually follow the same principle as that of human medical science.

### SUMMARY

The present disclosure relates to anti-canine PD-1 antibodies directed to canine PD-1. The antibodies have high binding affinity to canine PD-1, and have the ability to block the binding of PD-1 to PD-L1 of canine. In specific embodiments, the anti-canine PD-1 antibodies of the present disclosure are mouse-anti-canine PD-1 antibodies. In specific embodiments, the anti-canine PD-1 antibodies of the present disclosure are mouse-anti-canine PD-1 monoclonal antibodies.

The present disclosure further relates to functional fragments of an anti-canine PD-1 antibody directed to canine PD-1. The functional fragments of the anti-canine PD-1 antibody have high binding affinity to canine PD-1, and have the ability to block the binding of PD-1 to PD-L1 of canine.

The present disclosure further relates to a method for the preparation of the anti-canine PD-1 antibodies.

In a first aspect, the present disclosure provides an anti-canine PD-1 antibody capable of binding with canine PD-1; the anti-canine PD-1 antibody comprises three light-chain complementary determining regions (CDR1-3) or a conservatively modified variant maintaining the functions thereof; and/or three heavy-chain complementary determining regions (CDR1-3) or a conservatively modified variant maintaining functions thereof. In one embodiment, the light-chain complementary determining region CDR1 is selected from a group consisting of: TCAAGTGTAAGTTAC (SEQ ID NO: 1), TCAAGTGTAAGTTAC (SEQ ID NO: 2) and TCAAGTATAACTTAC (SEQ ID NO: 3); the light-chain complementary determining region CDR2 is: GACACATCC (SEQ ID NO: 4); the light-chain complementary determining region CDR3 is selected from a group consisting of: CAGCAGTACAGTGGTCACCCATCCTCG (SEQ ID NO: 5), CAACAGTACAGTGGTTACCCGTACACG (SEQ ID NO: 6) and CAGCAGTACAGTG GTTACCCATCCACG (SEQ ID NO: 7); the heavy-chain complementary determining region CDR1 is selected from a group consisting of: GGCTTCAACATCAAAGACACCTAT (SEQ ID NO: 8) and GGCTTCAA CATTAAAGACACCTAT (SEQ ID NO: 9); the heavy-chain complementary determining region CDR2 is selected from a group consisting of: ATTGATCCTGCGATTGATAATACT (SEQ ID NO: 10), ATTGATCCTGC GATTGGTAATACT (SEQ ID NO: 11) and ATTGATCCTGCGATTGGTAATCCT (SEQ ID NO: 12); the heavy-chain complementary determining region CDR3 is selected from a group consisting of: GCTTCTGGGTTCTATACTATGGACTAC (SEQ ID NO: 13), GCTAGAGGGTTCTATGGTATGGACTAC (SEQ ID NO: 14) and GCTTCTGGGTTCTATGCTATGGACTGC (SEQ ID NO: 15).

In one embodiment, the anti-canine PD-1 antibody comprises three light-chain complementary determining regions (CDR1-3) or a conservatively modified variant maintaining functions thereof, and three heavy-chain complementary determining regions (CDR1-3) or a conservatively modified variant maintaining functions thereof; the light-chain complementary determining region CDR1 is selected from a group consisting of: TCAAGTGTAACTTAC (SEQ ID NO: 1), TCAAGTGTAAGTTAC (SEQ ID NO: 2) and TCAAGTATAACTTAC (SEQ ID NO: 3); the light-chain complementary determining region CDR2 is: GACACATCC (SEQ ID NO: 4); the light-chain complementary determining region CDR3 is selected from a group consisting of: CAGCAGTACAGTGGTCACCCATCCTCG (SEQ ID NO: 5), CAACAGTACAGTGGTTACCCGTACACG (SEQ ID NO: 6) and CAGCAGTACAGTGGTTACCCATCCACG (SEQ ID NO: 7); the heavy-chain complementary determining region CDR1 is selected from a group consisting of: GGCTTCAACATCAAAGACACCTAT (SEQ ID NO: 8) and GGCTTCAACATTAAAGACACCTAT (SEQ ID NO: 9); the heavy-chain complementary determining region CDR2 is selected from a group consisting of: ATTGATCCTGCGATTGATAATACT (SEQ ID NO: 10), ATTGATCCTGCGATTGGTAATACT (SEQ ID NO: 11) and ATTGATCCTGCGATTGGTAATCCT (SEQ ID NO: 12); the heavy-chain complementary determining region CDR3 is selected from a group consisting of: GCTTCTGGGTTCTATACTATGGACTAC (SEQ ID NO: 13), GCTAGAGGGTTCTATGGTATGGACTAC (SEQ ID NO: 14) and GCTTCTG GGTTCTATGCTATGGACTGC (SEQ ID NO: 15).

In one embodiment, the anti-canine PD-1 antibody comprises the following combination of the three light-chain complementary determining regions (CDR1-3): (1) CDR1: TCAAGTGTAACTTAC (SEQ ID NO: 1), CDR2: GACACATCC (SEQ ID NO: 4), CDR3: CAGCAGTACAG TGGTCACCCATCCTCG (SEQ ID NO: 5); (2) CDR1: TCAAGTGTAAGTTAC (SEQ ID NO: 2), CDR2: GACACATCC (SEQ ID NO: 4), CDR3: CAACA GTACAGTGGTTACCCGTACACG (SEQ ID NO: 6); or (3) CDR1: TCAAG TATAACTTAC (SEQ ID NO: 3), CDR2: GACACATCC (SEQ ID NO: 4), CDR3: CAGCAGTACAGTGGTTACCCATCCACG (SEQ ID NO: 7).

In one embodiment, the anti-canine PD-1 antibody comprises the following combination of the three heavy-chain complementary determining regions (CDR1-3): (1) CDR1: GGCTTCAACATCAAAGACACCTAT (SEQ ID NO: 8), CDR2: ATTGATCCTGCGATTGATAATACT (SEQ ID NO: 10), CDR3: GCTTCTGGGTTCTATACTATGGACTAC (SEQ ID NO: 13); (2) CDR1: GGCTTCAACATTAAAGACACCTAT (SEQ ID NO: 9), CDR2: ATTGATCCTGCGATTGGTAATACT (SEQ ID NO: 11), CDR3: GCTTCTGGGTTCTATACTATGGACTAC (SEQ ID NO: 13); or (3) CDR1: GGCTTCAACATTAAAGACACCTAT (SEQ ID NO: 9), CDR2: ATTGATCCT GCGATTGGTAATCCT (SEQ ID NO: 12), CDR3: GCTTCTGGGTTCTATGCTATGGACTGC (SEQ ID NO: 15).

In one embodiment, the anti-canine PD-1 antibody is selected from the following combinations of the following three light-chain complementary determining regions (CDR1-3) and three heavy-chain complementary determining regions (CDR1-3): (1) light chain CDR1: TCAAGTGTAACTTAC (SEQ ID NO: 1), light chain CDR2: GACACATCC (SEQ ID NO: 4), light chain CDR3: CAGCAGTACAGTGGTCACCCATCCTCG (SEQ ID NO: 5); heavy chain CDR1: GGCTTCAACATCAAAGACACCTAT (SEQ ID NO: 8), heavy chain CDR2: ATTGATCCTGCGATTGATAATACT (SEQ ID NO: 10), heavy chain CDR3: GCTTCTGGGTTCTATACTATGGACTAC (SEQ ID NO: 13); (2) light chain CDR1: TCAAGTGTAAGTTAC (SEQ ID NO: 2), light chain CDR2: GACACATCC (SEQ ID NO: 4), light chain CDR3: CAACAGTACAGTGGTTACCC GTACACG (SEQ ID NO: 6); heavy chain CDR1: GGCTTCAACATTAAAGACACC TAT (SEQ ID NO: 9), heavy chain CDR2: ATTGATCCTGCGATTGGTAATACT (SEQ ID NO: 11), heavy chain CDR3: GCTTCTGGGTTCTATACTATGGACTAC (SEQ ID NO: 13); or (3) light chain CDR1: TCAAGTATAACTTAC (SEQ ID NO: 3), light chain CDR2: GACACATCC (SEQ ID NO: 4), light chain CDR3: CAGCAGTACAGTGGTTACCCATCCACG (SEQ ID NO: 7); heavy chain CDR1: GGCTTCAACATTAAAGACACCTAT (SEQ ID NO: 9), heavy chain CDR2: ATTGATCCTGCGATTGGTA ATCCT (SEQ ID NO: 12), heavy chain CDR3: GCTTCTGGGTTCTATGCTATGGAC TGC (SEQ ID NO: 15).

In one embodiment, the anti-canine PD-1 antibody comprises a light chain variable region selected from a group consisting of: (1) GCAAATTGTTCTCACCCAGTCTCCAGCAATCATGTCTGCATCTCCAGGGGAGAAGGTCACCATGA CCTGCAGTGCCAGCTCAAGTGTAACTTACATGTATTGGTTCCAGCAGAAGCCAGGCTCCTCCCCC AGACTCTGGATTTATGACACATCCAACCTGGTTTCTGGAGTCCCTGCTCGCTTCAGTGGCAGTAG GTCTGGGACCTCTTATTCTCTCACAATCAGCATATGGAGGCTGAAGATGCTGCCACTTATTACTGC CAGCAGTACAGTGGTCACCCATCCTCGTTCGGCTCGGGGACAAAGTTGGAAATTAAA (SEQ ID NO: 16); (2) CAAATTGTTCTCACCCAGTCTCCAGCAATCATGTCTGCATCTCCAGGGGAGAAGGTCACCATGAC CTGCAGTGCCAGCTCAAGTGTAAGTTACATGTTCTGGTACCAGCAGAAGCCAGGCTCCTCCCCC AGACTCTGGATTTATGACACATCCAACCTGGTTTCTGGAGTCCCTGCTCGCTTCAGTGGCAGTAG GTCTGGGACCTCTTATTCTCTCACAATCAGCAGCATGGAGGCTGAAGATGCTGCCACTTATTACT GCCAACAGTACAGTGGTTACCCGTACACGTTCGGAGGGGGGACCAAGCTGGAAATAAAG (SEQ ID NO: 17); and (3) CAAATTGTTCTCACCCAGTCTCCAGCCATCATGTCTGCATCTCCAGGGGAAAAGGTCACCATGAC CTGCAGTGCCAGCTCAAGTATAACTTACATGTTCTGGTACCAGCAGAAGCCAGGCTCCTCCCCCA GACTCTGGATTTATGACACATCCAACCTGGTTTCTGGAGTCCCTGCTCGCTTCAGTGGCAGTAAG TCTGGGACCTCTTATTCTCTCACAATCACCAGCATGGAGGCTGAAGATGCTGCCACTTATTACTGC CAGCAGTACAGTGGTTACCCATCCACGTTCGGCTCGGGGACAAAGTTGGAAATAAAA (SEQ ID NO: 18).

In one embodiment, the anti-canine PD-1 antibody comprises a heavy chain variable region nucleotide sequence selected from a group consisting of: (1) GAGGTTCAGCTGCAGCAGTCTGGGGCAGAGCTTGTGAAGCCAGGGGCCTCAGTCAAGTTGTCC TGCACAGCTTCTGGCTTCAACATCAAAGACACCTATATGCATTGGGTGAAGCAGAGGCCTGAAC AGGGCCTGGAGTGGATTGGAAGGATTGATCCTGCGATTGATAATACTAAATATGACCCGAAGTTC CAGGGCAAGGCCACTATAACAGCTGACACATCCTCCAACACAGCCTACCTGCAGCTCAGCAGCC TGACATCTGAGGACACTGCCGTCTATTACTGTGCTTCTGGGTTCTATACTATGGACTACTGGGGTC AAGGAACCTCAGTCACCGTCTCCTCA (SEQ ID NO: 19); (2) GAGGTTCGGCT GCAGCAGTCTGGGGCAGAGCTTGTGAAGCCAGGGGCCTCAGTCAAGTTGTCCTGCACAGCTTCT GGCTTCAACATTAAAGACACCTATTTACACTGGTTGAAGGAGAGGCCTGAACAGGGCCTGGAGT GGATTGGAAGGATTGATCCTGCGATTGGTAATACTAGATATGACCCGAAGTTCCAGGTCAAGGCC ACTATAACAGCAGACACATCCTCCAACACAGCCTACCTGCAACTCAGCAGCCTGACATCTGAGG ACTCTGCCGTCTATTACTGTGCTAGAGGGTTCTATGGTATGGACTACTGGGGTCAAGGAACCTCA GTCACCGTCTCCTCA (SEQ ID NO: 20); and (3) GAGGTTCAGCTGCAGCAGTCTGGGGCAGAGGTTGTGAAGCCAGGGGCCTCAGTCAAGTTGTCC TGCACAGCTTCTGGCTTCAACATTAAAGACACCTATATGCACTGGGTGAAGCAGAGGCCTGAAC AGGGCCTGGAGTGGATTGGAAGGATTGATCCTGCGATTGGTAATCCTAAATATGACCCGAAGTTC CAGGGCAGGGCCACTATAACTGCTGACACATCCTCCAACACAGCCTACCTGCAGCTCAGCAGCC TGACATCTGAGGACACTGCCGTCTATTACTGTGCTTCTGGGTTCTATGCTATGGACTGCTGGGGTC AAGGAACCTCAGTCACCGTCTCCTCA (SEQ ID NO: 21).

In one embodiment, the anti-canine PD-1 antibody comprises the following combination of the light chain variable regions and heavy chain variable regions: (1) a light chain variable region as shown by SEQ ID NO: 16 and a heavy chain variable region as shown by SEQ ID NO: 19; (2) a light chain variable region as shown by SEQ ID NO: 17 and a heavy chain variable region as shown by SEQ ID NO: 20; or (3) a light chain variable region as shown by SEQ ID NO: 18 and a heavy chain variable region as shown by SEQ ID NO: 21.

SEQ ID NO: 16 corresponds to the following amino acid sequence:

(SEQ ID NO: 22)
QIVLTQSPAIMSASPGEKVTMTCSASSSVTYMYWFQQKPGSSPRLWIYDT
SNLVSGVPARFSGSRSGTSYSLTISSMEAEDAATYYCQQYSGHPSSFGSG
TKLEIK.

SEQ ID NO: 17 corresponds to the following amino acid sequence:

(SEQ ID NO: 23)
QIVLTQSPAIMSASPGEKVTMTCSASSSVSYMFWYQQKPGSSPRLWIYDT
SNLVSGVPARFSGSRSGTSYSLTISSMEAEDAATYYCQQYSGYPYTFGGG
TKLEIK.

SEQ ID NO: 18 corresponds to the following amino acid sequence:

(SEQ ID NO: 24)
QIVLTQSPAIMSASPGEKVTMTCSASSSVSYMFWYQQKPGSSPRLWIYDT
SNLVSGVPARFSGSRSGTSYSLTISSMEAEDAATYYCQQYSGYPYTFGGG
TKLEIK.

SEQ ID NO: 19 corresponds to the following amino acid sequence:

(SEQ ID NO: 25)
EVQLQQSGAELVKPGASVKLSCTASGFNIKDTYMHWVKQRPEQGLEWIGR
IDPAIDNTKYDPKFQGKATITADTSSNTAYLQLSSLTSEDTAVYYCASGF
YTMDYWGQGTSVTVSS.

SEQ ID NO: 20 corresponds to the following amino acid sequence:

(SEQ ID NO: 26)
EVRLQQSGAELVKPGASVKLSCTASGFNIKDTYLHWLKERPEQGLEWIGR
IDPAIGNTRYDPKFQVKATITADTSSNTAYLQLSSLTSEDSAVYYCARGF
YGMDYWGQGTSVTVSS.

SEQ ID NO: 21 corresponds to the following amino acid sequence:

(SEQ ID NO: 27)
EVQLQQSGAEVVKPGASVKLSCTASGFNIKDTYMHWVKQRPEQGLEWIGR
IDPAIGNPKYDPKFQGRATITADTSSNTAYLQLSSLTSEDTAVYYCASGF
YAMDCWGQGTSVTVSS.

Preferably, the anti-canine PD-1 antibody is a mouse-anti-canine PD-1 antibody. Preferably, the anti-canine PD-1 antibody is a mouse-anti-canine PD-1 monoclonal antibody. Preferably, the anti-canine PD-1 antibody may block the binding of canine PD-1 to canine PD-L1.

In one embodiment, the anti-canine PD-1 antibody has an affinity of 1.000 E⁻⁰⁷-1.000 E⁻¹².

In one embodiment, the anti-canine PD-1 antibody blocks the binding of canine PD-1 to canine PD-L1; and the anti-canine PD-1 antibody has an affinity of 1.000 E⁻⁰⁷-1.000 E⁻¹².

In one embodiment, the anti-canine PD-1 antibody is a caninized anti-canine PD-1 monoclonal antibody, and the caninized anti-canine PD-1 monoclonal antibody may block the binding of the canine PD-1 to canine PD-L1.

In one embodiment, the caninized anti-canine PD-1 monoclonal antibody comprises a heavy chain constant region and/or a light chain constant region of the canine PD-1 monoclonal antibody.

The heavy chain constant region has a sequence as shown by SEQ ID NO: 28, and the light chain constant region has a sequence as shown by SEQ ID NO: 29.

In a second aspect, the present disclosure provides a functional fragment of an anti-canine PD-1 antibody capable of binding with canine PD-1, comprising three light-chain complementary determining regions (CDR1-3) or a combination of conservatively modified variants maintaining the functions thereof, or comprising three heavy-chain complementary determining regions (CDR1-3) or a combination of conservatively modified variants maintaining functions thereof; the light-chain complementary determining region CDR1 is selected from a group consisting of: TCAAGTGTAACTTAC (SEQ ID NO: 1), TCAAGTGTAAGTTAC (SEQ ID NO: 2) and TCAAGTATAACTTAC (SEQ ID NO: 3); the light-chain complementary determining region CDR2 is: GACACATCC (SEQ ID NO: 4); the light-chain complementary determining region CDR3 is selected from a group consisting of: CAGCAGTACAGTGGTCACCCATCCTCG (SEQ ID NO: 5), CAACAGTACAGTGGTTACCCGTACACG (SEQ ID NO: 6) and CAGCAGTACAGTGGTTACCCATCCACG (SEQ ID NO: 7); the heavy-chain complementary determining region CDR1 is selected from a group consisting of: GGCTTCAACATCAAAGACACCTAT (SEQ ID NO: 8) and GGCTTCAACATTAAAGACACCTAT (SEQ ID NO: 9); the heavy-chain complementary determining region CDR2 is selected from a group consisting of: ATTGATCCTGCGATTGATAATACT (SEQ ID NO: 10), ATTGATCCTGCGATTGGTAATACT (SEQ ID NO: 11) and ATTGATCCTGCGATTGGTAATCCT (SEQ ID NO: 12); the heavy-chain complementary determining region CDR3 is selected from a group consisting of: GCTTCTGGGTTCTATACTATGGACTAC (SEQ ID NO: 13), GCTAGAGGGTTCTATGGTATGGACTAC (SEQ ID NO: 14) and GCTTCTG GGTTCTATGCTATGGACTGC (SEQ ID NO: 15).

In one embodiment, the functional fragment of the anti-canine PD-1 antibody comprises the following combination of the three light-chain complementary determining regions (CDR1-3): (1) CDR1: TCAAGTGTAACTTAC (SEQ ID NO: 1), CDR2: GACACATCC (SEQ ID NO: 4), CDR3: CAGCAGTACAGTGGTCACCCATCCTCG (SEQ ID NO: 5); (2) CDR1: TCAAGTGTAAGTTAC (SEQ ID NO: 2), CDR2: GACACATCC (SEQ ID NO: 4), CDR3: CAACAGTACAGTGGTTACCCGTACACG (SEQ ID NO: 6); or (3) CDR1: TCAAGTATAACTTAC (SEQ ID NO: 3), CDR2: GACACATCC (SEQ ID NO: 4), CDR3: CAGCAGTACAGTGGTTACCCATCCACG (SEQ ID NO: 7),

or a combination of the conservatively modified variant maintaining functions thereof.

In one embodiment, the functional fragment of the anti-canine PD-1 antibody comprises the following combination of the three heavy-chain complementary determining regions (CDR1-3): (1) CDR1: GGCTTCAACATCAAAGACACCTAT (SEQ ID NO: 8), CDR2: ATTGATCCTGCGATTGATAATACT (SEQ ID NO: 10), CDR3: GCTTCTGGGTTCTATACTATGGACTAC (SEQ ID NO: 13); (2) CDR1: GGCTTCAACATTAAAGACACCTAT (SEQ ID NO: 9), CDR2: ATTGATCCTGCGATTGGTAATACT (SEQ ID NO: 11), CDR3: GCTTCTGGGTTCTATACTATGGACTAC (SEQ ID NO: 13); or (3) CDR1: GGCTTCAACATTAAAGACACCTAT (SEQ ID NO: 9), CDR2: ATTGATCCTGCGATTGGTAATCCT (SEQ ID NO: 12), CDR3: GCTTCTGGGTTCTATGC TATGGACTGC (SEQ ID NO: 15), or a combination of the conservatively modified variant maintaining functions thereof.

In one embodiment, the functional fragment of the anti-canine PD-1 antibody comprises the following combinations of the three light-chain complementary determining regions (CDR1-3) and three heavy-chain complementary determining regions (CDR1-3): (1) light chain CDR1: TCAAGTGTAACTTAC (SEQ ID NO: 1), light chain CDR2: GACACATCC (SEQ ID NO: 4), light chain CDR3: CAGCAGTACAGTGGTCACCCATCCTCG (SEQ ID NO: 5), heavy chain CDR1: GGCTTCAACATCAAAGACACCTAT (SEQ ID NO: 8), heavy chain CDR2: ATTGATCCTGCGATTGATAATACT (SEQ ID NO: 10), heavy chain CDR3: GCTTCTGGGTTCTATACTATGGACTAC (SEQ ID NO: 13); (2) light chain CDR1: TCAAGTGTAAGTTAC (SEQ ID NO: 2), light chain CDR2: GACACATCC (SEQ ID NO: 4), light chain CDR3: CAACAGTACAGTGGTTACCCGTACACG (SEQ ID NO: 6), heavy chain CDR1: GGCTTCAACATTAAAGACACCTAT (SEQ ID NO: 9), heavy chain CDR2: ATTGATCCTGCGATTGGTAATACT (SEQ ID NO: 11), heavy chain CDR3: GCTTCTGGGTTCTATACTATGGACTAC (SEQ ID NO: 13); or (3) light chain CDR1: TCAAGTATAACTTAC (SEQ ID NO: 3), light chain CDR2: GACACATCC (SEQ ID NO: 4), light chain CDR3: CAGCAGTACAGTGGTTACCCATCCACG (SEQ ID NO: 7), heavy chain CDR1: GGCTTCAACATTAAAGACACCTAT (SEQ ID NO: 9), heavy chain CDR2: ATTGATCCTGCGATTGGTA ATCCT (SEQ ID NO: 12), heavy chain CDR3: GCTTCTGGGTTCTATGCTATGGACTGC (SEQ ID NO: 15),

or a combination of the conservatively modified variant maintaining functions thereof.

In one embodiment, the anti-canine PD-1 antibody comprises the following combination of the three light-chain complementary determining regions (CDR1-3) and three heavy-chain complementary determining regions (CDR1-3): (1) light chain CDR1: TCAAGTGTAACTTAC (SEQ ID NO: 1), light chain CDR2: GACACATCC (SEQ ID NO: 4), light chain CDR3: CAGCAGTACAGTGG TCACCCATCCTCG (SEQ ID NO: 5), heavy chain CDR1: GGCTTCAACATCAAAGACACCTAT (SEQ ID NO: 8), heavy chain CDR2: ATTGATCCTGCGATTGATAATACT (SEQ ID NO: 10), heavy chain CDR3: GCTTCTGGGTTCTATACTATGGACTAC (SEQ ID NO: 13); (2) light chain CDR1: TCAAGTGTAAGTTAC (SEQ ID NO: 2), light chain CDR2: GACACATCC (SEQ ID NO: 4), light chain CDR3: CAACAGTACAGTGGTTACCCGTACACG (SEQ ID NO: 6), heavy chain CDR1: GGCTTCAACATTAAAGACACCTAT (SEQ ID NO: 9), heavy chain CDR2: ATTGA TCCTGCGATTGGTAATACT (SEQ ID NO: 11), heavy chain CDR3: GCTTCTGGGTT CTATACTATGGACTAC (SEQ ID NO: 13); or (3) light chain CDR1: TCAAGTATAA CTTAC (SEQ ID NO: 3), light chain CDR2: GACACATCC (SEQ ID NO: 4), light chain CDR3: CAGCAGTACAGTGGTTACCCATCCACG (SEQ ID NO: 7), heavy chain CDR1: GGCTTCAACATTAAAGACACCTAT (SEQ ID NO: 9), heavy chain CDR2: ATTG ATCCTGCGATTGGTAATCCT (SEQ ID NO: 12), heavy chain CDR3: GCTTCTGGG TTCTATGCTATGGACTGC (SEQ ID NO: 15).

In one embodiment, the functional fragment of the anti-canine PD-1 antibody comprises a light chain variable region nucleotide sequence selected from a group consisting of: (1) a nucleotide sequence as shown by SEQ ID NO: 16; (2) a nucleotide sequence as shown by SEQ ID NO: 17; and (3) a nucleotide sequence as shown by SEQ ID NO: 18.

In one embodiment, the functional fragment of the anti-canine PD-1 antibody comprises a heavy chain variable region nucleotide sequence selected from a group consisting of: (1) a nucleotide sequence as shown by SEQ ID NO: 19; (2) a nucleotide sequence as shown by SEQ ID NO: 20; and (3) a nucleotide sequence as shown by SEQ ID NO: 21.

In one embodiment, the functional fragment of the anti-canine PD-1 antibody comprises the following combination of the light chain variable regions and heavy chain variable regions: (1) a light chain variable region as shown by SEQ ID NO: 16 and a heavy chain variable region as shown by SEQ ID NO: 19; (2) a light chain variable region as shown by SEQ ID NO: 17 and a heavy chain variable region as shown by SEQ ID NO: 20; or (3) a light chain variable region as shown by SEQ ID NO: 18 and a heavy chain variable region as shown by SEQ ID NO: 21,

or a combination of the conservatively modified variant maintaining functions thereof.

Preferably, the functional fragment is an antigen-binding fragment.

Abbreviations and key terms are defined as follows:

Programmed cell death receptor 1 (PD-1) is an important immunosuppressive molecule, and belongs to an immune globulin superfamily. PD-1 is a membrane protein containing 268 amino acid residues. PD-1 is initially cloned from apoptotic mice T-cell hybridoma 2B4.11. Immunoregulation targeting PD-1 is of great significance in anti-tumor, anti-infection, anti-autoimmune disease, organ grafting survival and the like.

PD-1 is an inhibitory T cell receptor which mainly works in tumor microenvironment through two known ligands programmed cell death ligand 1 (PD-L1, also called B7-H1 or CD274) and programmed cell death ligand 2 (PD-L2, also called B7-DC or CD273). PD-L1 may also serve as a target. The antibody of PD-L1 may play the same role as that of PD-1. The binding of PD-1 to PD-L1 initiates the programmed death of T cells, resulting in tumor immune escape. Therefore, PD-1 and PD-L1 play an important role in the anti-tumor immunologic process of an organism.

PD-1 is selectively increased in response to the immunosuppressive signal directly transmitted by cancer, and PD-1 plays a role in regulating major action factors. In the field of human medical science, the latest evidences from preclinical models emphasize the key functions of PD-1, as a T-cell co-receptor, and its ligands B7-H1/PD-L1 and B7-DC/PD-L2 in maintaining the microenvironment of immunosuppressive tumors.

The binding of PD-1 and PD-L1/PD-L2 can recruit a tyrosine phosphatases SHP-2 via an immunoreceptor tyrosine-based switch motif ITSM, leading to dephosphorylating a plurality of key molecules on the TCR signaling pathway, inhibiting the activization of native T cells and functions of effector T cells, inducing the production of regulatory T cells and maintaining the inhibitory functions of regulatory T cells. Moreover, PD-L1 expressed on the surface of antigen-presenting cells can bind to CD80 on T cells, thereby inhibiting immune response. In the micro-environment of tumor, the activated PD-1/PD-L1 signaling pathway can reduce tumor-specific T cell immune effect, thereby mediating tumor immune escape and accelerating tumor growth.

The specific antitumor mechanism may comprise the following aspects:

(1) PD-L1 on the surface of tumor cells and PD-1 on the surface of tumor-specific T cells are bound to induce tolerance, apoptosis and depletion of T cells;

(2) activated PD-1 can selectively inhibit RAS/MEK/ERK and PI3K/AKT signaling pathways, inhibit the transcription and protein expression of cell cycle-related genes, and hinder cell cycle, thereby inhibiting the proliferation of T cells; and

(3) PD-L1 on the surface of antigen-presenting cells can accelerate the conversion of CD4 T cells into induced Treg (iTreg) with maintaining the inhibitory function thereof, and further inhibit the activity of effector T cells, by down-regulating the phosphorylation level of signal molecules mTOR, AKT, S6 and ERK2 in CD4 T cells and up-regulating PTEN expression.

Furthermore, Duraiswamy, et al. also found that the depletion of tumor infiltrating lymphocytes (TILs) in tumor microenvironment was closely related to PD-L1 expressed by tumor cells and myeloid-derived suppressor cells (tumor-associated macrophages, dendritic cells and the like). The blocking of PD-1/PD-L1 signaling pathway can enhance the functions of effector CD8 T cells, and inhibit the functions of the Treg cells and myeloid-derived suppressor cells, thereby enhancing the anti-tumor effect of immune system.

Antibodies against PD-1 or PD-L1 restore T cell activity by blocking the binding of PD-1 to PD-L1, and exert the anti-tumor effect.

Canine cancers are good spontaneous cancer comparison models, and the therapeutic methods for the canine cancers usually follow the same principle as that of human medical science. Some studies have molecularly identified canine PD-1 and PD-L1, and discussed the potential of canine PD-1 and PD-L1 as a therapeutic target of canine tumors. Studies have found that PD-L1 is only expressed on the cell surface of a small amount of tumor cell lines, but expressed inside the cells of almost all cell lines. Moreover, PD-L1 is highly frequently expressed in malignant melanoma, breast tumors, mastocytoma and hemangiosarcoma of canine. Canine tumor samples are subjected to flow cytometry, western blotting and immunohistochemistry. The results show that canine mastocytoma, malignant melanoma, breast tumors, and soft tissue sarcoma seem to be good tumor candidates which can be treated by PD-1 and PD-L1 antibodies. Therefore, the anti-canine PD-1 antibodies of the present disclosure and functional fragments thereof can serve as candidate drugs for treating canine tumors, and can enhance the anti-tumor effect of canine immune system and exert the effector functions as therapeutic antibodies. The present disclosure provides very effective and broad-spectrum therapeutical method for canine cancers.

The “conservatively modified variant” refers that amino acids having similar properties are used to substitute the corresponding amino acids in a certain protein, and the modified protein has the same bioactivity as the unmodified protein. The following table shows exemplary conservative substitutes for the amino acids:

| Amino | Conservatively substituted |
|---|---|
| acid residues | amino acid residues |
| Ala(A) | Gly; Ser |
| Arg(R) | Lys; His |
| Asn(N) | Gln; His |
| Asp(D) | Glu; Asn |
| Cys(C) | Ser; Ala |
| Gln (Q) | Asn |
| Glu(E) | Asp; Gln |
| Gly(G) | Ala |
| His(H) | Asn; Gln |
| Ile(I) | Leu; Val |
| Leu(L) | Ile; Val |
| Lys(K) | Arg; His |
| Met(M) | Leu; Ile; Tyr |
| Phe(F) | Tyr; Met; Leu |
| Pro(P) | Ala; Gly |
| Ser(S) | Thr |
| Thr(T) | Ser |
| Trp(W) | Tyr; Phe |
| Tyr(Y) | Trp; Phe |
| Val(V) | Ile; Leu. |

### BRIEF DESCRIPTION OF THE DRAWINGS

FIG. 1 shows the SDS-PAGE electrophoresis pattern and western blotting pattern obtained by using an antigen fragment which was expressed by expressing a vector in Expi293F cells and purified.

FIG. 2 shows the ELISA experimental results of six mice from two groups 21 days after immunization.

FIG. 3 shows the ELISA detection results of the immunoreaction between the supernatant from two hybridoma libraries and canine PD-1.

FIG. 4 shows the binding determined over time.

FIGS. 5-9 show the responses of 30 antibodies from Nos. M1 to M30 to PD-1 His over time.

FIG. 10 shows the binding determined over time during the measurement.

FIGS. 11-12 show the test results of M1-M for the analyte PD1 Fc.

FIG. 13 shows that the binding of the antibodies C1, C3 and C11 of the present disclosure to PBMC do not influence the proliferation of lymphocyte.

FIG. 14 shows that the antibodies C1, C3 and C11 of the present disclosure can be effectively bound to PD1 on the surface of T cells.

FIG. 15 shows that the addition of the antibodies C1, C3 and C11 of the present disclosure does not influence the release of interferon.

### DETAILED DESCRIPTION OF THE EMBODIMENTS

The technical solution of the present disclosure will be further described in combination with examples and accompanying drawings of the description. These examples are merely used to describe the present disclosure, but not intent to limit the protection scope of the present disclosure.

### EXAMPLES

### Experimental Materials and Methods:

1. Plasmid Preparation
1) design a target sequence, optimize and synthesize;
2) subclone the complete sequence into pcDNA3.1 plasmid; and
3) prepare a large number of plasmids required by transfecting Expi293F cells for expression.

2. Cell Culture and Transient Transfection
1) culture Expi293F cells in a serum-free Expi293 expression medium;
2) put cell culture flasks into an incubator at 37° C. and 8% CO₂ for shaking culture, and the day before transfection, inoculate the cells into Corning cell culture flasks with proper density;
3) mix DNA and transfection reagents in an optimal proportion on the day of transfection, and then add the mixed solution into the culture flask for cell transfection;
4) transiently transfect the plasmids encoding the target recombinant protein to suspension Expi293F cell culture; and
5) collect the cell culture supernatant on the 6th day for purification.

3. Purification and Analysis
1) centrifuge the cell culture;
2) slowly add the supernatant to an affinity purification column;
3) use proper buffer solution for cleaning and elution, combine multiple eluates and change the buffer solution to the final formulation to prepare a buffer solution;
4) subject the purified protein to SDS-PAGE and Western blotting analysis to measure the molecular weight and purity; and
5) measure the protein concentration by means of the BCA method using BSA as a standard.

4. Preparation and Screening of Mouse-Anti-Canine PD-1 Monoclonal Antibody
1) rapidly immunize mice, 3 Balb/C mice in each group, and two groups in total;
2) detect serum antigen titers from all the 6 mice by means of ELISA on day 21;
3) rapidly immunize mice, fusion, select hybridomas;
4) detect antigen titers in the supernatant of hybridoma cells by using ELISA;
5) screen the supernatant of hybridoma cell by using flow cytometry;
6) culture mice monoclonal antibodies, 1 cell/well, thirty 96-well plates in total;
7) detect antigen titers of the mice monoclonal antibodies by using ELISA, ten plates in total for screening;
8) select monoclonal antibodies for amplification and freeze preservation;
9) perform dynamics affinity analysis on mice monoclonal antibody supernatant; and
10) perform dynamics cross-block analysis on the mice monoclonal antibody supernatant.

5. PBMC Separation
Reagents: Lymphoprep™, PBS and RPMI-1640
Experimental process:
1) collect 5 mL canine peripheral blood with a heparin sodium-treated anticoagulant tube;
2) add an equal volume of PBS buffer solution and mix with canine anticoagulation;
3) add 5 mL lymphocyte separation solution in a density of 1.077 g/mL into a 15 mL centrifugal tube, and add 5 mL blood diluent into the centrifugal tube, centrifuge at 800 g for 20 min at room temperature;
4) gently insert a capillary pipet into an albuginea layer on the interface, carefully withdraw the middle leucocyte layer, add an equal amount of RPMI-1640, mix well, centrifuge at 800 g for 20 min at room temperature, and discard supernatant; and
5) resuspend with RPMI-1640, centrifuge at 800 g for 20 min at room temperature, and discard supernatant; then repeat the above steps once.

6. PBMC Culture
1) count PBMC obtained from the lymphocyte separation solution and culture in a 12-well plate at the conditions of 10% FBS, 37° C. and 5% CO₂;
2) divide PBMC into two groups, in which one group was added with ConA at a final concentration of 0.5 μg/mL and the other group was without ConA; and
3) optionally, divide the group added with ConA into several treatment groups, for example, for the addition of different antibodies, according to the requirements of the experiment.

7. PBMC PD-1 Flow Cytometry Detection
Antibodies: CD3-FITC; M1, M3 and M11 monoclonal antibodies; and PE-labeled anti-canine IgG antibody
1) count ConA-activated and nonactivated PBMC, respectively, take 5×10 E3 cells respectively and resuspend in 100 μL PBS;
2) stain PBMC with M1, M3, M11 and CD3-FITC respectively, including non-staining tubes, single staining tubes, and double staining tubes; incubate the antibodies in dark place for 30 min at room temperature;
3) centrifuge cells at 200 g for 5 min, and discard supernatant, resuspend the cells with 100 μL PBS, add the PE-labeled anti-canine IgG antibody and incubate in dark place for 20 min at room temperature; and
4) centrifuge cells at 200 g for 5 min, discard supernatant, and resuspend the cells with 500 μL PBS, and detect on a flow cytometer.

### Example 1. Preparation of Antigens and Standards

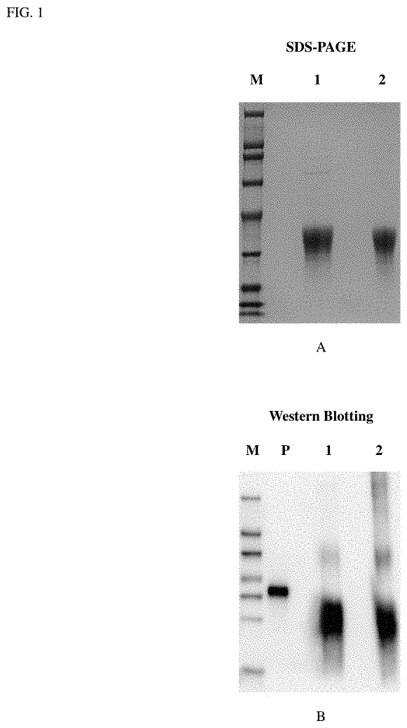

The amino acids 25-168 of the canine PD-1 protein were picked, and the former signal peptide was removed. This amino acid sequence was add with a His tag at the terminal end for purification and detection. A gene fragment encoding this amino acid sequence was synthesized and cloned into a pUC57 plasmid for preservation, and then subcloned into pcDNA 3.1 plasmid. A large number of transfection plasmids were prepared for the expression in Expi293F cells. Expi293F cells were maintained in suspension culture, and transiently transfected with the plasmids. Then the expressed protein was purified and analyzed for the concentration. Results are shown in FIGS. 1A and 1B. FIG. 1A shows SDS-PAGE electrophoresis pattern, in which lane M is protein markers corresponding to 200 kD, 116 kD, 971d), 66 kD, 44 kD, 29 kD, 20 kD, 14 kD and 6 kD respectively; lane 1 shows purified proteins under reduction conditions; lane 2 shows purified proteins under non-reduction conditions. FIG. 1A shows Western Blotting pattern, in which lane M is protein markers corresponding to 120 kD, 80 kD, 60 kD, 50 kD, 421d), 321(1) and 18 kD respectively; lane P shows a His-tag protein positive control; lane 1 shows purified proteins under reduction conditions; lane 2 shows purified proteins under non-reduction conditions. The primary antibody: mouse-anti-His mAb.

Optimization of codon or not: Codon optimization was performed
Expression system and vector:
   Mammal, and expression vector pcDNA3.1
Host cell line: Expi293F
Optimization of the expression or not:
   Expression optimization was performed
Expression system: 40 mL serum-free Expi293F™ expression medium
Protein were obtained from the supernatant of cell culture, and further Purification: purified by HisTrap™ FF Crude
Concentration: 2.4 mg/mL, determined by BCA™ (BSA used as standard)
Purity: About 95%, Coomassie blue-stained SDS-PAGE gel was subjected to densitometric analysis for estimation under non-reduction conditions
Sterilization: Filtered by a 0.22 μm filter and aseptic packaging was performed
Storage: Preserved at −80° C., subpackaged and freeze preserved to avoid repeated freeze thawing
Storage of the buffer solution: 1×PBS, pH 7.2

### Example 2. Preparation of Monoclonal Antibodies

The protein prepared in Example 1 was used as an antigen to immunize mice rapidly, 3 mice in each group, and two groups in total. On day 21, the serum antigen titers were determined for the immunized mice, fused and selected to build a hybridoma library. Supernatant antigen titers of the hybridoma library were determined and screened with flow cytometry. Then monoclonal antibodies were selected for culture, amplified and froze for preservation. Finally, the dynamics affinity and dynamics cross-block analysis were performed for the mice monoclonal antibody supernatant.

1. Description of the Parameters for Analyzing the Serum from the Mice on Day 21:
Coat EIA/RIA polystyrene plates with antigen--->block--->primary antibody--->detection antibody--->substrate--->stop and read.
Analysis type: a solid phase antigen ELISA
Ag ID: PD-1 His
Ab ID: PD-1A mice serum, D21 m1-m3 Ab
Ab dilution: 1/1000
Ab ID: PD-1S mice serum, D21 m1-m3 Ab
Ab dilution: 1/1000
Ab ID: normal mice serum Ab
Ab dilution: 1/1000
Detection reagent ID: goat-anti-mouse HRP
Dilution: 1/5000

TABLE 1
Test results of the serum of mice on day 21

| | | | | | | | Normal |
|---|---|---|---|---|---|---|---|
| | | | | | | | mice |
| Ag | PD-1A | PD-1A | PD-1A | PD-1s | PD-1s | PD-1s | serum |
| (ng/mL) | m1 | m2 | m3 | m1 | m2 | m3 | Control |
| 1000 | 3.45 | 3.43 | 3.38 | 3.24 | 3.45 | 3.36 | 0.18 |
| 333 | 3.37 | 3.31 | 3.4 | 2.59 | 3.29 | 2.98 | 0.14 |
| 111 | 3.22 | 2.44 | 2.53 | 1.09 | 2.22 | 1.32 | 0.12 |
| 37 | 2.26 | 1 | 1.21 | 0.48 | 0.83 | 0.53 | 0.11 |
| 12 | 0.8 | 0.28 | 0.34 | 0.18 | 0.22 | 0.2 | 0.08 |
| 4 | 0.35 | 0.16 | 0.19 | 0.13 | 0.14 | 0.14 | 0.07 |
| 1 | 0.18 | 0.1 | 0.11 | 0.11 | 0.09 | 0.09 | 0.09 |
| No | 0.1 | 0.12 | 0.07 | 0.09 | 0.07 | 0.11 | 0.08 |

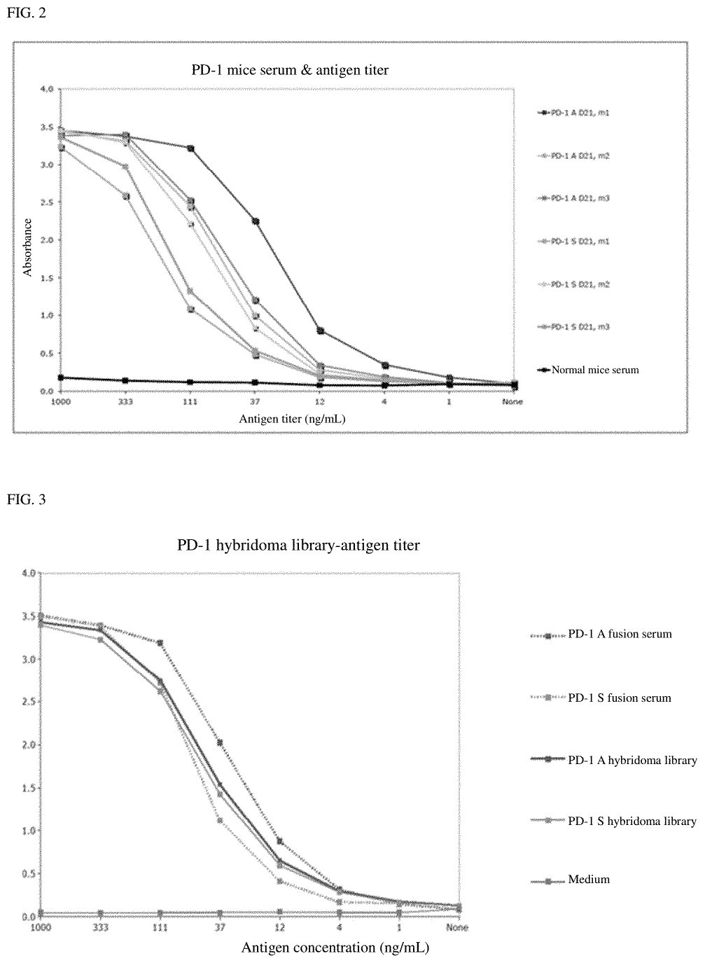

Furthermore, referring to FIG. 2, it can be seen from the ELISA experimental results that all 6 mice of the two groups produce good antibody responses 21 days after immunization, and can be subjected to the next experiment.

2. Description of the Parameters of the Immunoassay for the Supernatant of the Hybridoma Library
Coat EIA/RIA polystyrene plates with antigen--->block--->primary antibody--->detection antibody--->substrate--->stop and read.
Analysis type: a solid phase antigen ELISA
Ag ID: PD-1 His
Ab ID: PD-1A mice serum, D21 m1-m3 Ab
Ab dilution: 1/1000
Ab ID: PD-1S mice serum, D21 m1-m3 Ab
Ab dilution: 1/1000
Ab ID: PD-1A hybridoma library
Ab dilution: undiluted
Ab ID: PD-1S hybridoma library
Ab dilution: undiluted
Detection reagent ID: goat-anti-mouse HRP
Dilution: 1/5000

TABLE 2
Immunoassay results of the supernatant of the hybridoma library

| Ag | PD-1 A | | PD-1 A | PD-1 S | |
|---|---|---|---|---|---|
| Concentration | fused | PD-1 S | hybridoma | hybridoma | |
| (ng/mL) | serum | fused serum | library | library | Medium |
| 1000 | 3.51 | 3.50 | 3.43 | 3.40 | 0.05 |
| 333 | 3.39 | 3.39 | 3.34 | 3.23 | 0.04 |
| 111 | 3.19 | 2.72 | 2.75 | 2.62 | 0.04 |
| 37 | 2.02 | 1.12 | 1.54 | 1.43 | 0.05 |
| 12 | 0.88 | 0.41 | 0.65 | 0.59 | 0.06 |
| 4 | 0.31 | 0.17 | 0.30 | 0.28 | 0.05 |
| 1 | 0.14 | 0.15 | 0.17 | 0.16 | 0.05 |
| No | 0.08 | 0.08 | 0.13 | 0.12 | 0.09 |

Moreover, referring to FIG. 3, it can be seen from the ELISA results that the supernatants from two hybridoma libraries have good immunoreactions to canine PD-1. Next, monoclonal antibodies can be generated by cloning. The antibodies were cloned in 30 plates of 96-well in total, and each well had a single cell. The single cell was cultured, amplified and preserved. ELISA was performed on the supernatant of each cell culture, so as to screen out monoclonal antibodies having strong activities against canine PD-1.

3. Screening of the Monoclonal Antibodies:
Test type: a solid phase antigen ELISA
Ag ID: PD-1 His
Ag concentration/dilution: 100 ng/mL
Ag ID: PD-1A mice serum, D21 m1-m3
Ab concentration/dilution: 1/1000
Ab ID: PD-1A hybridoma library
Ab concentration/dilution: undiluted
Ab ID: PD-1A McAb supernatant
Detection reagent ID: goat-anti-mouse HRP-Fc
Dilution: 1/5000

After ELISA, 30 cell lines were selected, which had good immunoreactions and were likely to produce different antibodies, and renumbered from M1 to M30 as shown by Table 3 below. The antibodies produced by these cell lines were used for the following testing and screening.

TABLE 3
Screening results of the monoclonal antibodies

| | | O.D. determined by ELISA |
|---|---|---|
| Clone names | ELISA/T.C. vessel | compared with PD-1 His |
| PD-1 A-M1 | 2C08 | 2.65 |
| PD-1 A-M2 | 3G04 | 3.11 |
| PD-1 A-M3 | 4G01 | 2.87 |
| PD-1 A-M4 | 4G08 | 3.23 |
| PD-1 A-M5 | 5B05 | 3.11 |
| PD-1 A-M6 | 5F09 | 2.71 |
| PD-1 A-M7 | 6A10 | 2.57 |
| PD-1 A-M8 | 7H06 | 2.80 |
| PD-1 A-M9 | 8F02 | 3.09 |
| PD-1 A-M10 | 8H01 | 2.83 |
| PD-1 A-M11 | 9B04 | 2.66 |
| PD-1 A-M12 | 9E06 | 3.06 |
| PD-1 A-M13 | 10A04 | 2.74 |
| PD-1 A-M14 | 10G01 | 3.30 |
| PD-1 A-M15 | 11E10 | 2.34 |
| PD-1 A-M16 | 11F06 | 2.78 |
| PD-1 A-M17 | 13B03 | 3.00 |
| PD-1 A-M18 | 13C10 | 2.50 |
| PD-1 A-M19 | 13E11 | 2.87 |
| PD-1 A-M20 | 14A06 | 2.93 |
| PD-1 A-M21 | 15A03 | 2.89 |
| PD-1 A-M22 | 15G11 | 3.02 |
| PD-1 A-M23 | 18A08 | 2.58 |
| PD-1 A-M24 | 19B12 | 3.16 |
| PD-1 A-M25 | 21D02 | 2.54 |
| PD-1 A-M26 | 21G04 | 2.81 |
| PD-1 A-M27 | 23F10 | 2.64 |
| PD-1 A-M28 | 26F03 | 2.56 |
| PD-1 A-M29 | 29D02 | 3.03 |
| PD-1 A-M30 | 29E08 | 2.73 |
| Negative control | HT Medium | 0.07 |
| Positive control | Positive serum | 3.16 |
| Positive control | Hybridoma library | 3.25 |

4. Affinity Screening of the Monoclonal Antibodies:

Test Procedures:
Sensor detect (30 s)--->load Ab (700 s)--->negative quench (480 s)--->base line (300 s)--->Ab bind (600 s)-->dissociate (600 s)-->repeat

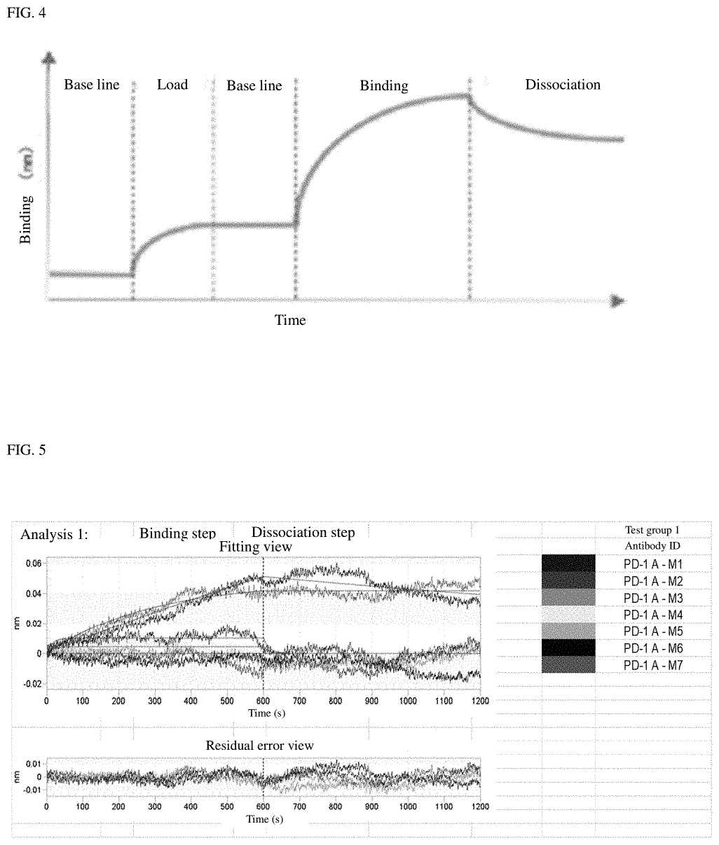

The binding or dissociation with the surface can cause migration, and thus the binding was determined by measuring the migration changes over time (see FIG. 4).

Octet BMIA was used to determine the affinity of the supernatant of the PD-1A clone culture to PD-1 His. Anti-mouse IgG Fc was added to each clone supernatant to capture competence factors.

The following clones show no PD-1 His binding:

PD-1A-M2
PD-1A-M4
PD-1A-M5
PD-1A-M6
PD-1A-M7
PD-1A-M9
PD-1A-M12
PD-1A-M13
PD-1A-M14
PD-1A-M16
PD-1 A-M18
PD-1 A-M19
PD-1 A-M20
PD-1 A-M21
PD-1 A-M24
PD-1 A-M25
PD-1 A-M26
PD-1 A-M28
PD-1 A-M29

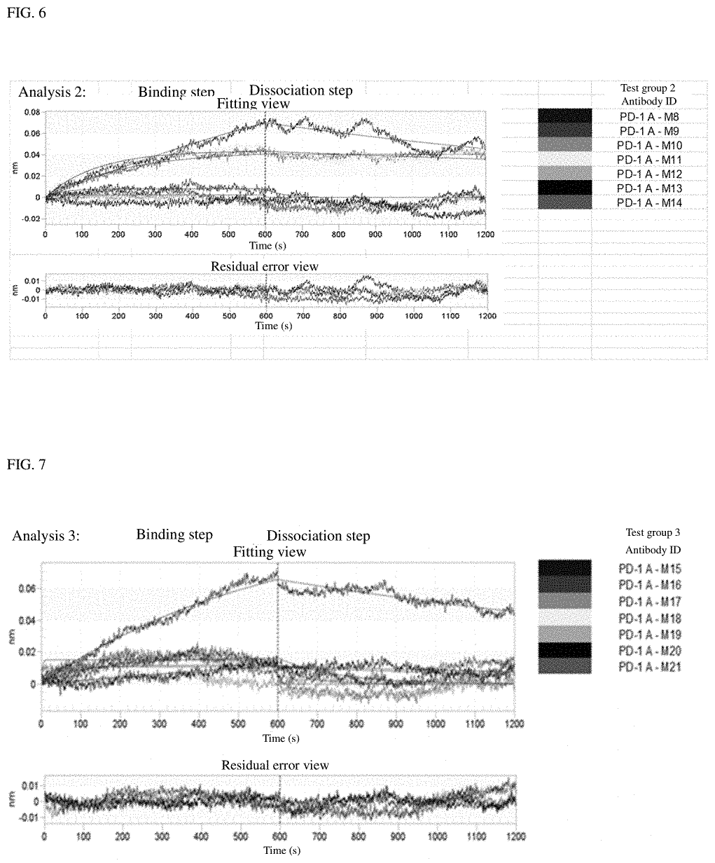

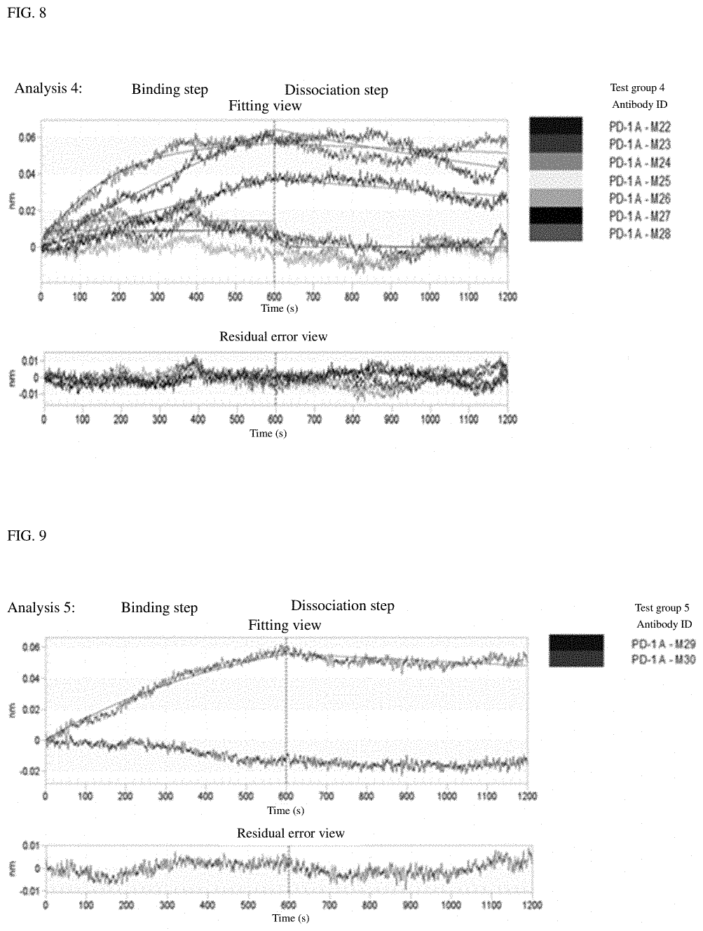

FIGS. 5-9 show the changes in the responses of 30 antibodies from Nos. M1 to M30 (M cAb supernatant amplified by undiluted PD-1A) to PD-1 His (a concentration of 150 μg/mL) over time.

TABLE 4
Sorting of the clone supernatants according to KDs and responses to PD-1 His

| | Analyte | | | | | |
|---|---|---|---|---|---|---|
| | concentration | Response | | | | |
| Antibody ID | (nM) | (nm) | K_{D} | kₒₙ | k_{dis} | R² |
| PD-1 A-M3 | 20 | 0.041 | <1.0E−12 | 1.740E+05 | <1.0E−07 | 0.921 |
| PD-1 A-M10 | 20 | 0.044 | 3.063E−11 | 2.272E+05 | 6.959E−06 | 0.914 |
| PD-1 A-M23 | 20 | 0.061 | 5.371E−10 | 2.957E+05 | 1.588E−04 | 0.900 |
| PD-1 A-M11 | 20 | 0.049 | 9.035E−10 | 3.456E+05 | 3.123E−04 | 0.827 |
| PD-1 A-M30 | 20 | 0.059 | 2.904E−09 | 8.543E+04 | 2.481E−04 | 0.964 |
| PD-1 A-M15 | 20 | 0.068 | 1.039E−08 | 5.851E+04 | 6.079E−04 | 0.964 |
| PD-1 A-M1 | 20 | 0.049 | 1.438E−07 | 3.059E+03 | 4.399E−04 | 0.925 |
| PD-1 A-M8 | 20 | 0.069 | 2.211E−07 | 3.231E+03 | 7.143E−04 | 0.918 |
| PD-1 A-M27 | 20 | 0.037 | 2.333E−07 | 2.042E+03 | 4.764E−04 | 0.922 |
| PD-1 A-M22 | 20 | 0.060 | 2.472E−07 | 2.700E+03 | 6.674E−04 | 0.949 |
| PD-1 A-M17 | 20 | 0.012 | 1.428E−05 | 2.458E+04 | 3.509E−01 | 0.800 |
| PD-1 A-M2 | 20 | −0.011 | — | — | — | — |
| PD-1 A-M4 | 20 | 0.002 | — | — | — | — |
| PD-1 A-MS | 20 | −0.005 | — | — | — | — |
| PD-1 A-M6 | 20 | −0.003 | — | — | — | — |
| PD-1 A-M7 | 20 | 0.005 | — | — | — | — |
| PD-1 A-M9 | 20 | −0.006 | — | — | — | — |
| PD-1 A-M12 | 20 | −0.005 | — | — | — | — |
| PD-1 A-M13 | 20 | −0.002 | — | — | — | — |
| PD-1 A-M14 | 20 | 0.006 | — | — | — | — |
| PD-1 A-M16 | 20 | 0.008 | — | — | — | — |
| PD-1 A-M18 | 20 | 0.011 | — | — | — | — |
| PD-1 A-M19 | 20 | 0.000 | — | — | — | — |
| PD-1 A-M20 | 20 | 0.012 | — | — | — | — |
| PD-1 A-M21 | 20 | 0.013 | — | — | — | — |
| PD-1 A-M24 | 20 | 0.010 | — | — | — | — |
| PD-1 A-M25 | 20 | 0.006 | — | — | — | — |
| PD-1 A-M26 | 20 | −0.004 | — | — | — | — |
| PD-1 A-M28 | 20 | 0.004 | — | — | — | — |
| PD-1 A-M29 | 20 | −0.012 | — | — | — | — |

Note:
response refers to the response (nm) calculated during the binding step;
K_{D} refers to an affinity constant (M = k_{dis}/kₒₙ)
kₒₙ refers to a binding rate (1/Ms)
k_{dis} refers to a dissociation rate (1/s)
Full-R² refers to a curve-fitting correlation coefficient

Thus, antibodies having high affinities for canine PD-1 were obtained, as shown by Table 5 below.

TABLE 5
Antibodies having high affinities for canine PD-1
PD-1 A-M3
PD-1 A-M10
PD-1 A-M23
PD-1 A-M11
PD-1 A-M30
PD-1 A-M15
PD-1 A-M1
PD-1 A-M8
PD-1 A-M27
PD-1 A-M22

5. Screening of the Block Function of the Monoclonal Antibodies
Preincubate of antibodies and antigens (1 h)--->balance (30 s)--->Load Lead Ab(600 s)--->quench (300 s)--->base line (300 s)-->Ab+Ag bind (300 s)-->repeat

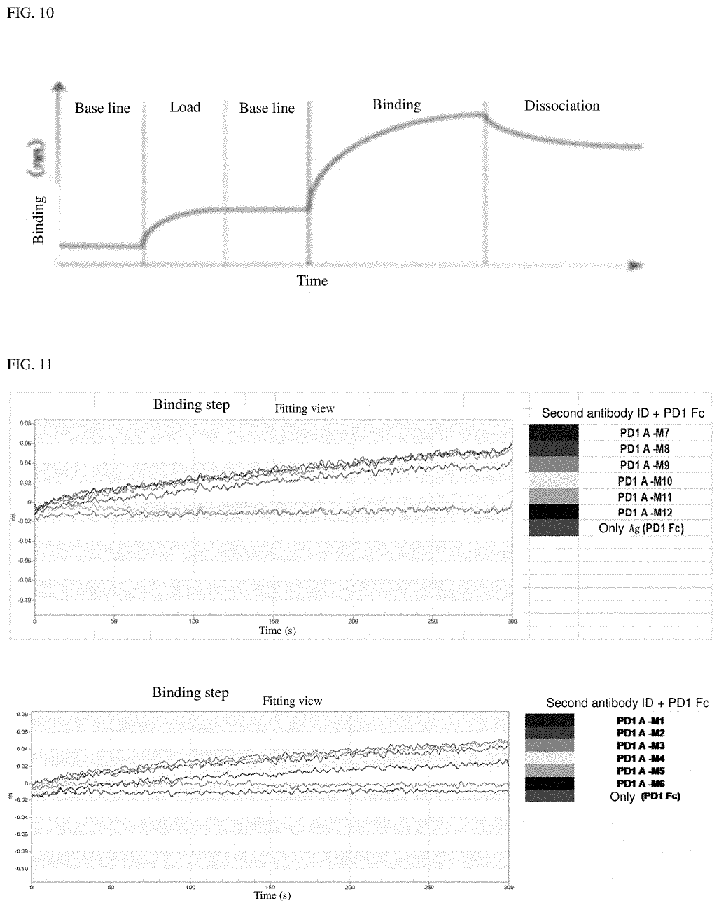

The binding or dissociation with the surface can cause migration, and thus the binding was determined by measuring the migration changes over time (see FIG. 10).

D-L1 Fc (5p g/mL) was loaded on an AHC sensor for cross-linking assay. Epitope analysis was used to determine the binding of antibodies loaded on the sensor to recombinant antibodies preincubated with PD1-Fc. The binding (response) degrees observed were compared to that of antigen only under the same conditions. If the total response of an antibody was higher than that of antigen only, the antibody would be matched. If the response of an antibody was lower than 80% of that of antigen, the antibody would be blocked with each other.

Sorted by the lowest to highest % of the response, the following clones blocking the binding of PD1-Fc to PD-L1 were obtained:
PD1 A-M27
PD1 A-M1
PD1 A-M22
PD1 A-M8
PD1 A-M11
PD1 A-M15
PD1 A-M3
PD1 A-M23
PD1 A-M10
PD1 A-M13
PD1 A-M21
PD1 A-M19
PD1 A-M18
PD1 A-M30
PD1 A-M24
PD1 A-M6
PD1 A-M16
PD1 A-M20
PD1 A-M7
PD1 A-M17

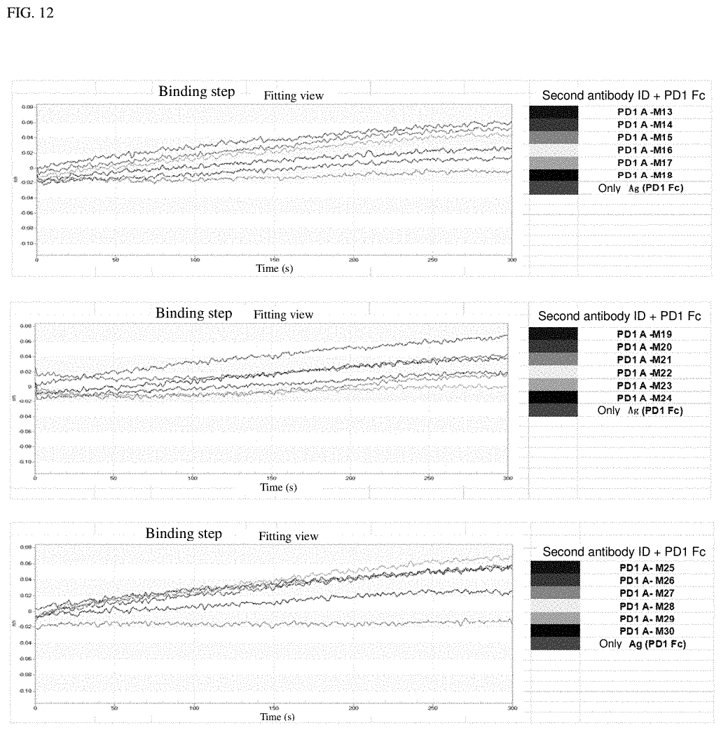

FIGS. 11-12 show the test results for which the loaded Ab is PD-L1 Fc, the concentration (μg/mL) is 5; Ab is PD1A McAb supernatant, the dilution ratio is ½, quenching Ab is human γ-immunoglobulin in a concentration (μg/mL) of 150, the analyte is PD1 Fc in a concentration (nM) of 50.

TABLE 6

| | | | | | Sorting of response by % | | | | |
|---|---|---|---|---|---|---|---|---|---|
| | | Response | | | (lowest to highest) | | | | |
| Lead Ab ID | Second Ab ID | (nm) | % response | Block/pair | Lead Ab ID | Secondary Ab ID | Response (nm) | % response | Block/pair |
| PD-Ll Fc | PD1 A - M1 | −0.009 | -21.90% | Block | PD-Ll Fc | PD1 A - M27 | −0.012 | −22.04% | Block |
| PD-Ll Fc | PD1 A - M2 | 0.050 | 118.57% | Pair | PD-Ll Fc | PD1 A - M1 | −0.009 | −21.90% | Block |
| PD-Ll Fc | PD1 A - M3 | −0.002 | -5.00% | Block | PD-Ll Fc | PD1 A - M22 | −0.013 | −20.53% | Block |
| PD-Ll Fc | PD1 A - M4 | 0.034 | 81.19% | N/A | PD-Ll Fc | PD1 A - M8 | −0.009 | −18.56% | Block |
| PD-Ll Fc | PD1 A - M5 | 0.047 | 111.19% | Pair | PD-Ll Fc | PD1 A - M11 | −0.008 | −15.97% | Block |
| PD-Ll Fc | PD1 A - M6 | 0.025 | 58.81% | Block | PD-Ll Fc | PD1 A - M15 | −0.005 | -8.21% | Block |
| PD-Ll Fc | Only Ag (PD1 Fc) | 0.042 | 100.00% | N/A | PD-Ll Fc | PD1 A - M3 | −0.002 | -5.00% | Block |
| PD-Ll Fc | PD1 A - M7 | 0.036 | 71.26% | Block | PD-Ll Fc | PD1 A - M23 | 0.000 | −0.47% | Block |
| PD-Ll Fc | PD1 A - M8 | −0.009 | −18.56% | Block | PD-Ll Fc | PD1 A - M10 | 0.006 | 11.98% | Block |
| PD-Ll Fc | PD1 A - M9 | 0.048 | 95.41% | N/A | PD-Ll Fc | PD1 A - M13 | 0.013 | 22.11% | Block |
| PD-Ll Fc | PD1 A - M10 | 0.006 | 11.98% | Block | PD-Ll Fc | PD1 A - M21 | 0.015 | 23.48% | Block |
| PD-Ll Fc | PD1 A - M11 | −0.008 | −15.97% | Block | PD-Ll Fc | PD1 A - M19 | 0.017 | 27.06% | Block |
| PD-Ll Fc | PD1 A - M12 | 0.052 | 102.99% | Pair | PD-Ll Fc | PD1 A - M18 | 0.025 | 42.38% | Block |
| PD-Ll Fc | Only Ag (PD1 Fc) | 0.050 | 100.00% | N/A | PD-Ll Fc | PD1 A - M30 | 0.023 | 43.15% | Block |
| PD-Ll Fc | PD1 A - M13 | 0.013 | 22.11% | Block | PD-Ll Fc | PD1 A - M24 | 0.037 | 57.23% | Block |
| PD-Ll Fc | PD1 A - M14 | 0.052 | 87.60% | N/A | PD-Ll Fc | PD1 A - M6 | 0.025 | 58.81% | Block |
| PD-Ll Fc | PD1 A - M15 | −0.005 | -8.21% | Block | PD-Ll Fc | PD1 A - M16 | 0.037 | 61.81% | Block |
| PD-Ll Fc | PD1 A - M16 | 0.037 | 61.81% | Block | PD-Ll Fc | PD1 A - M20 | 0.042 | 64.85% | Block |
| PD-Ll Fc | PD1 A - M17 | 0.044 | 74.20% | Block | PD-Ll Fc | PD1 A - M7 | 0.036 | 71.26% | Block |
| PD-Ll Fc | PD1 A - M18 | 0.025 | 42.38% | Block | PD-Ll Fc | PD1 A - M17 | 0.044 | 74.20% | Block |
| PD-Ll Fc | Only Ag (PD1 Fc) | 0.060 | 100.00% | N/A | PD-Ll Fc | PD1 A - M4 | 0.034 | 81.19% | N/A |
| PD-Ll Fc | PD1 A - M19 | 0.017 | 27.06% | Block | PD-Ll Fc | PD1 A - M14 | 0.052 | 87.60% | N/A |
| PD-Ll Fc | PD1 A - M20 | 0.042 | 64.85% | Block | PD-Ll Fc | PD1 A - M9 | 0.048 | 95.41% | N/A |
| PD-Ll Fc | PD1 A - M21 | 0.015 | 23.48% | Block | PD-Ll Fc | PD1 A - M26 | 0.053 | 98.70% | N/A |
| PD-Ll Fc | PD1 A - M22 | −0.013 | −20.53% | Block | PD-Ll Fc | PD1 A - M12 | 0.052 | 102.99% | Pair |
| PD-Ll Fc | PD1 A - M23 | 0.000 | −0.47% | Block | PD-Ll Fc | PD1 A - M25 | 0.056 | 104.44% | Pair |
| PD-Ll Fc | PD1 A - M24 | 0.037 | 57.23% | Block | PD-Ll Fc | PD1 A - M28 | 0.057 | 105.56% | Pair |
| PD-Ll Fc | Only Ag (PD1 Fc) | 0.064 | 100.00% | N/A | PD-Ll Fc | PD1 A - M5 | 0.047 | 111.19% | Pair |
| PD-Ll Fc | PD1 A - M25 | 0.056 | 104.44% | Pair | PD-Ll Fc | PD1 A - M2 | 0.050 | 118.57% | Pair |
| PD-Ll Fc | PD1 A - M26 | 0.053 | 98.70% | N/A | PD-Ll Fc | PD1 A - M29 | 0.067 | 124.26% | Pair |
| PD-Ll Fc | PD1 A - M27 | −0.012 | −22.04% | Block | PD-Ll Fc | Only Ag (PD1 Fc) | 0.042 | 100.00% | N/A |
| PD-Ll Fc | PD1 A - M28 | 0.057 | 105.56% | Pair | PD-Ll Fc | Only Ag (PD1 Fc) | 0.050 | 100.00% | N/A |
| PD-Ll Fc | PD1 A - M29 | 0.067 | 124.26% | Pair | PD-Ll Fc | Only Ag (PD1 Fc) | 0.060 | 100.00% | N/A |
| PD-Ll Fc | PD1 A - M30 | 0.023 | 43.15% | Block | PD-Ll Fc | Only Ag (PD1 Fc) | 0.064 | 100.00% | N/A |
| PD-Ll Fc | Only Ag (PD1 Fc) | 0.054 | 100.00% | N/A | PD-Ll Fc | Only Ag (PD1 Fc) | 0.054 | 100.00% | N/A |

### Abbreviation

Abbreviation:
Keywords: Response Response (nm) calculated during the binding step
   KD Affinity constant (M)=k_{dis}/kₒₙ
   kₒₙ Binding rate (1/Ms)
   k_{dis} Dissociation rate (1/s)
   Full-R² Curve-fitting correlation coefficient

The antibodies having strong ability to block the binding of canine PD-1 to PD L-1 were finally confirmed below:
PD1 A-M27
PD1 A-M1
PD1 A-M22
PD1 A-M8
PD1 A-M11
PD1 A-M15
PD1 A-M3
PD1 A-M23
PD1 A-M10
PD1 A-M13

6. Final Screening of the Monoclonal Antibodies

The following 10 monoclonal antibodies were selected for antibody subtype tests, according to the affinity test results and block function screen results.

TABLE 7

| | | | Antibody light |
|---|---|---|---|
| Monoclonal antibody | Affinity | Block Function | chain subtype |
| M3 | <1.000E⁻¹² | −5.00% | IgG1 kappa |
| M10 | 3.063E⁻¹¹ | 11.98% | IgG1 kappa |
| M23 | 5.371E⁻¹⁰ | −0.47% | IgG1 κ |
| M11 | 9.035E⁻¹⁰ | −15.97% | IgG1 κ |
| M13 | / | 22.11% | IgG2b κ |
| M15 | 1.039E⁻⁰⁸ | −8.21% | IgG1 κ |
| M1 | 1.438E⁻⁰⁷ | −21.90% | IgG1 κ |
| M8 | 2.211E⁻⁰⁷ | −18.56% | IgG1 κ |
| M27 | 2.333E⁻⁰⁷ | −22.04% | IgG1 κ |
| M22 | 2.472E−07 | −20.53% | IgG1 κ |

Then, six cell lines were selected from the 10 antibodies for DNA sequence analysis. The selected 6 antibodies were M1, M3, M10, M11, M15 and M23.

### Example 3. Determination, Caninization and Expression of the Monoclonal Antibodies

(1) Sequence Determination of the Monoclonal Antibodies
1) extract RNA after the centrifugation and precipitation of hybridoma cell lines;
2) perform reverse transcription RT-PCR (5′RACE) and amplification of the light- and heavy chain variable regions of the hybridoma antibodies;
3) clone DNA fragments produced by PCR into a sequencing plasmids and transform into Escherichia coli;
4) pick 6-12 bacterial colonies and extract plasmid DNA, respectively;
5) design primer sequences for the plasmid to sequence the cloned DNA fragments;
6) determine the common sequences shared by several bacterial colones; and
7) perform analysis to obtain the cDNA sequences of the antibody variable regions.

(2) cDNA Sequences of the Variable Regions of the Mouse-Anti-Canine PD-1 Monoclonal Antibodies

1) Protein Sequences in the Variable Regions of the Selected 6 Antibodies

| | |
|---|---|
| M1K | QIVLTQSPAIMSASPEKVTMTCSASSSVTYMYWFQQKPGSSPRLWIYDTSNLVSGVPARFSGSRSGTSYSLTISS |
| | MEAEDAATYYCQQYSGHPSSFGSGTKLEIK |
| M3K | QIVLTQSPAIMSASPEKVTMTCSASSSVSYMFWYQQKPGSSPRLWIYDTSNLVSGVPARFSGSRSGTSYSLTISS |
| | MEAEDAATYYCQQYSGHPSSFGSGTKLEIK |
| M10K | QIVLTQSPAIMSASPEKVTMTCSASSSVSYMFWYQQKPGSSPRLWIYDTSNLVSGVPARFSGSRSGTSYSLTISS |
| | MEAEDAATYYCQQYSGHPSSFGSGTKLEIK |
| M11K | QIVLTQSPAIMSASPEKVTMTCSASSSVTYMFWYQQKPGSSPRLWIYDTSNLVSGVPARFSGSRSGTSYSLTISS |
| | MEAEDAATYYCQQYSGHPSSFGSGTKLEIK |
| M15K | QIVLTQSPAIMSASPEKVTMTCSASSSVSYMFWYQQKPGSSPRLWIYDTSNLVSGVPARFSGSRSGTSYSLTISS |
| | MEAEDAATYYCQQYSGHPSSFGSGTKLEIK |
| M23K | QIVLTQSPAIMSASPEKVTMTCSASSSVSYMFWYQQKPGSSPRLWIYDTSNLVSGVPARFSGSRSGTSYSLTISS |
| | MEAEDAATYYCQQYSGHPSSFGSGTKLEIK |
| M1H | EVQLQQSGAELVKPGASVKLSCTASGFNIKDTYMHWVKQRPEQLEWIGRIDPAIDNTKYDPKFQGKATITADTSS |
| | NTAYQLSSTSEDTAVYYCASGFYTMDYWGQGTSVTVSS |
| M3H | EVRLQQSGAELVKPGASVKLSCTASGFNIKDTYLHWLKERPEQLEWIGRIDPAIGNTRYDPKFQVKATITADTSS |
| | NTAYQLSSTSEDSAVYYCARGFYGMDYWGQGTSVTVSS |
| M10H | EVRLQQSGAELVKPGASVKLSCTASGFNIKDTYLHWLKERPEQLEWIGRIDPAIGNTRYDPKFQVKATITADTSS |
| | NTAYQLSSTSEDSAVYYCARGFYGMDYWGQGTSVTVSS |
| M11H | EVQLQQSGAEVVKPGASVKLSCTASGFNIKDTYMHWVKQRPEQLEWIGRIDPAIDNPKYDPKFQGKATITADTSS |
| | NTAYQLSSTSEDTAVYYCASGFYAMDCWGQGTSVTVSS |
| M15H | EVRLQQSGAELVKPGASVKLSCTASGFNIKDTYLHWLKERPEQLEWIGRIDPAIDNTRYDPKFQVKATITADTSS |
| | NTAYQLSSTSEDSAVYYCARGFYGMDYWGQGTSVTVSS |
| M23H | EVRLQQSGAELVKPGASVKLSCTASGFNIKDTYLHWLKERPEQLEWIGRIDPAIDNTRYDPKFQVKATITADTSS |
| | NTAYQLSSTSEDSAVYYCARGFYGMDYWGQGTSVTVSS |

The selected 6 antibodies were M1, M3, M10, M11, M15 and M23. K: light chain, H: heavy chain.

(2) cDNA Sequences of the Variable Regions of the Selected 6 Antibodies

M1K:
GCAAATTGTTCTCACCCAGTCTCCAGCAATCATGTCTGCATCTCCAGGGGAGAAGGTCACCA
TGACCTGCAGTGCCAGCTCAAGTGTAACTTACATGTATTGGTTCCAGCAGAAGCCAGGCTCC
TCCCCCAGACTCTGGATTTATGACACATCCAACCTGGTTTCTGGAGTCCCTGCTCGCTTCAG
TGGCAGTAGGTCTGGGACCTCTTATTCTCTCACAATCAGCATATGGAGGCTGAAGATGCTGC
CACTTATTACTGCCAGCAGTACAGTGGTCACCCATCCTCGTTCGGCTCGGGGACAAAGTTGG
AAATTAAA
M3K:
CAAATTGTTCTCACCCAGTCTCCAGCAATCATGTCTGCATCTCCAGGGGAGAAGGTCACCAT
GACCTGCAGTGCCAGCTCAAGTGTAAGTTACATGTTCTGGTACCAGCAGAAGCCAGGCTCCT
CCCCCAGACTCTGGATTTATGACACATCCAACCTGGTTTCTGGAGTCCCTGCTCGCTTCAGT
GGCAGTAGGTCTGGGACCTCTTATTCTCTCACAATCAGCAGCATGGAGGCTGAAGATGCTGC
CACTTATTACTGCCAACAGTACAGTGGTTACCCGTACACGTTCGGAGGGGGGACCAAGCTGG
AAATAAAG
M10K:
CAAATTGTTCTCACCCAGTCTCCAGCAATCATGTCTGCATCTCCAGGGGAGAAGGTCACCAT
GACCTGCAGTGCCAGCTCAAGTGTAAGTTACATGTTCTGGTACCAGCAGAAGCCAGGCTCCT
CCCCCAGACTCTGGATTTATGACACATCCAACCTGGTTTCTGGAGTCCCTGCTCGCTTCAGT
GGCAGTAGGTCTGGGACCTCTTATTCTCTCACAATCAGCAGCATGGAGGCTGAAGATGCTGC
CACTTATTACTGCCAACAGTACAGTGGTTACCCGTACACGTTCGGAGGGGGGACCAAGCTGG
AAATAAAG
M11K:
CAAATTGTTCTCACCCAGTCTCCAGCCATCATGTCTGCATCTCCAGGGGAAAAGGTCACCAT
GACCTGCAGTGCCAGCTCAAGTATAACTTACATGTTCTGGTACCAGCAGAAGCCAGGCTCCT
CCCCCAGACTCTGGATTTATGACACATCCAACCTGGTTTCTGGAGTCCCTGCTCGCTTCAGT
GGCAGTAAGTCTGGGACCTCTTATTCTCTCACAATCACCAGCATGGAGGCTGAAGATGCTGC
CACTTATTACTGCCAGCAGTACAGTGGTTACCCATCCACGTTCGGCTCGGGGACAAAGTTGG
AAATAAAA
M15K:
CAAATTGTTCTCACCCAGTCTCCAGCAATCATGTCTGCATCTCCAGGGGAGAAGGTCACCAT
GACCTGCAGTGCCAGCTCAAGTGTAAGTTACATGTTCTGGTACCAGCAGAAGCCAGGCTCCT
CCCCCAGACTCTGGATTTATGACACATCCAACCTGGTTTCTGGAGTCCCTGCTCGCTTCAGT
GGCAGTAGGTCTGGGACCTCTTATTCTCTCACAATCAGCAGCATGGAGGCTGAAGATGCTGC
CACTTATTACTGCCAACAGTACAGTGGTTACCCGTACACGTTCGGAGGGGGGACCAAGCTGG
AAATAAAG
M23K:
CAAATTGTTCTCACCCAGTCTCCAGCAATCATGTCTGCATCTCCAGGGGAGAAGGTCACCAT
GACCTGCAGTGCCAGCTCAAGTGTAAGTTACATGTTCTGGTACCAGCAGAAGCCAGGCTCCT
CCCCCAGACTCTGGATTTATGACACATCCAACCTGGTTTCTGGAGTCCCTGCTCGCTTCAGT
GGCAGTAGGTCTGGGACCTCTTATTCTCTCACAATCAGCAGCATGGAGGCTGAAGATGCTGC
CACTTATTACTGCCAACAGTACAGTGGTTACCCGTACACGTTCGGAGGGGGGACCAAGCTGG
AAATAAAG
M1H:
GAGGTTCAGCTGCAGCAGTCTGGGGCAGAGCTTGTGAAGCCAGGGGCCTCAGTCAAGTTGTC
CTGCACAGCTTCTGGCTTCAACATCAAAGACACCTATATGCATTGGGTGAAGCAGAGGCCTG
AACAGGGCCTGGAGTGGATTGGAAGGATTGATCCTGCGATTGATAATACTAAATATGACCCG
AAGTTCCAGGGCAAGGCCACTATAACAGCTGACACATCCTCCAACACAGCCTACCTGCAGCT
CAGCAGCCTGACATCTGAGGACACTGCCGTCTATTACTGTGCTTCTGGGTTCTATACTATGG
ACTACTGGGGTCAAGGAACCTCAGTCACCGTCTCCTCA
M3H:
GAGGTTCGGCTGCAGCAGTCTGGGGCAGAGCTTGTGAAGCCAGGGGCCTCAGTCAAGTTGTC
CTGCACAGCTTCTGGCTTCAACATTAAAGACACCTATTTACACTGGTTGAAGGAGAGGCCTG
AACAGGGCCTGGAGTGGATTGGAAGGATTGATCCTGCGATTGGTAATACTAGATATGACCCG
AAGTTCCAGGTCAAGGCCACTATAACAGCAGACACATCCTCCAACACAGCCTACCTGCAACT
CAGCAGCCTGACATCTGAGGACTCTGCCGTCTATTACTGTGCTAGAGGGTTCTATGGTATGG
ACTACTGGGGTCAAGGAACCTCAGTCACCGTCTCCTCA
M10H:
GAGGTTCGGCTGCAGCAGTCTGGGGCAGAGCTTGTGAAGCCAGGGGCCTCAGTCAAGTTGTC
CTGCACAGCTTCTGGCTTCAACATTAAAGACACCTATTTACACTGGTTGAAGGAGAGGCCTG
AACAGGGCCTGGAGTGGATTGGAAGGATTGATCCTGCGATTGGTAATACTAGATATGACCCG
AAGTTCCAGGTCAAGGCCACTATAACAGCAGACACATCCTCCAACACAGCCTACCTGCAACT
CAGCAGCCTGACATCTGAGGACTCTGCCGTCTATTACTGTGCTAGAGGGTTCTATGGTATGG
ACTACTGGGGTCAAGGAACCTCAGTCACCGTCTCCTCA
M11H:
GAGGTTCAGCTGCAGCAGTCTGGGGCAGAGGTTGTGAAGCCAGGGGCCTCAGTCAAGTTGTC
CTGCACAGCTTCTGGCTTCAACATTAAAGACACCTATATGCACTGGGTGAAGCAGAGGCCTG
AACAGGGCCTGGAGTGGATTGGAAGGATTGATCCTGCGATTGGTAATCCTAAATATGACCCG
AAGTTCCAGGGCAGGGCCACTATAACTGCTGACACATCCTCCAACACAGCCTACCTGCAGCT
CAGCAGCCTGACATCTGAGGACACTGCCGTCTATTACTGTGCTTCTGGGTTCTATGCTATGG
ACTGCTGGGGTCAAGGAACCTCAGTCACCGTCTCCTCA
M15H:
GAGGTTCGGCTGCAGCAGTCTGGGGCAGAGCTTGTGAAGCCAGGGGCCTCAGTCAAGTTGTC
CTGCACAGCTTCTGGCTTCAACATTAAAGACACCTATTTACACTGGTTGAAGGAGAGGCCTG
AACAGGGCCTGGAGTGGATTGGAAGGATTGATCCTGCGATTGGTAATACTAGATATGACCCG
AAGTTCCAGGTCAAGGCCACTATAACAGCAGACACATCCTCCAACACAGCCTACCTGCAACT
CAGCAGCCTGACATCTGAGGACTCTGCCGTCTATTACTGTGCTAGAGGGTTCTATGGTATGG
ACTACTGGGGTCAAGGAACCTCAGTCACCGTCTCCTCA
M23H:
GAGGTTCGGCTGCAGCAGTCTGGGGCAGAGCTTGTGAAGCCAGGGGCCTCAGTCAAGTTGTC
CTGCACAGCTTCTGGCTTCAACATTAAAGACACCTATTTACACTGGTTGAAGGAGAGGCCTG
AACAGGGCCTGGAGTGGATTGGAAGGATTGATCCTGCGATTGGTAATACTAGATATGACCCG
AAGTTCCAGGTCAAGGCCACTATAACAGCAGACACATCCTCCAACACAGCCTACCTGCAACT
CAGCAGCCTGACATCTGAGGACTCTGCCGTCTATTACTGTGCTAGAGGGTTCTATGGTATGG
ACTACTGGGGTCAAGGAACCTCAGTCACCGTCTCCTCA

(3) Sequencing Result Analysis on the Selected Antibodies

Based on the antibody sequencing results, it was found that, in the 6 antibodies, M3, M10, M15 and M23 were the same antibody. Therefore, the finally confirmed antibodies were M1, M3 and M11.

(4) Caninization and Affinity Experimental Results of the Monoclonal Antibodies:

Heavy chain constant regions and light chain constant regions of the three monoclonal antibodies M1, M3 and M11 were replaced with a heavy chain constant region (as shown by SEQ ID NO: 28) and a light chain constant region (as shown by SEQ ID NO: 29) of the canine PD-1 monoclonal antibody. The resulted caninized monoclonal antibodies were respectively named C1, C3 and C11. C1 corresponds to the monoclonal antibody (NO. 35.38) in the table below; C3 corresponds to the monoclonal antibody (NO. 36.39) in the table below; and C11 corresponds to the monoclonal antibody (NO. 37.40) in Table 8 below.

TABLE 8

| Antibody ID | K_{D} | kₒₙ | k_{dis} | R² |
|---|---|---|---|---|
| C1(35.38) | 1.595E⁻⁰⁹ | 2.512E⁺⁰⁸ | 4.007E⁻⁰¹ | 0.778 |
| C3(36.39) | <1.0E⁻¹² | 6.265E⁺⁰² | <1.0E⁻⁰⁷ | 0.714 |
| C11(37.40) | <1.0E⁻¹² | 1.190⁺⁰³ | <1.0E⁻⁰⁷ | 0.667 |

TABLE 9

| | PD-1 His | Response | | | | |
|---|---|---|---|---|---|---|
| Antibody ID | Conc. (nM) | (nm) | K_{D} | kₒₙ | k_{dis} | R² |
| 35.38 | 1000.0 | 0.001 | — | — | — | — |
| 35.38 | 333.3 | −0.004 | — | — | — | — |
| 35.38 | 111.3 | 0.016 | 2.060E−09 | 4.300E+05 | 8.859E−04 | 0.377 |
| 35.38 | 37.3 | 0.008 | 2.647E−09 | 3.599E+06 | 9.528E−03 | 0.819 |
| 35.38 | 12.4 | 0.001 | 2.456E−09 | 8.046E+06 | 1.976E−02 | 0.380 |
| 35.38 | 4.0 | 0.005 | 1.835E−08 | 6.932E+07 | 1.272E+00 | 0.843 |
| 35.38 | 1.1 | −0.031 | — | — | — | — |
| | | Global Fit: | 1.595E−09 | 2.512E+08 | 4.007E−01 | 0.778 |
| 36.39 | 1000.0 | 0.007 | <1.0E−12 | 3.296E+02 | <1.0E−07 | 0.334 |
| 36.39 | 333.3 | 0.010 | <1.0E−12 | 1.765E+03 | <1.0E−07 | 0.661 |
| 36.39 | 111.3 | 0.026 | <1.0E−12 | 1.470E+04 | <1.0E−07 | 0.804 |
| 36.39 | 37.3 | 0.017 | <1.0E−12 | 4.051E+04 | <1.0E−07 | 0.887 |
| 36.39 | 12.4 | 0.005 | — | — | — | — |
| 36.39 | 4.0 | 0.009 | — | — | — | — |
| 36.39 | 1.1 | 0.002 | — | — | — | — |
| | | Global Fit: | <1.0E−12 | 6.265E+02 | <1.0E−07 | 0.714 |
| 37.40 | 1000.0 | 0.009 | <1.0E−12 | 1.474E+03 | <1.0E−07 | 0.368 |
| 37.40 | 333.3 | 0.012 | <1.0E−12 | 9.474E+02 | <1.0E−07 | 0.838 |
| 37.40 | 111.3 | 0.025 | <1.0E−12 | 2.375E+04 | <1.0E−07 | 0.715 |
| 37.40 | 37.3 | 0.015 | <1.0E−12 | 6.755E+04 | <1.0E−07 | 0.703 |
| 37.40 | 12.4 | 0.008 | <1.0E−12 | 5.010E+04 | <1.0E−07 | 0.588 |
| 37.40 | 4.0 | 0.004 | — | — | — | — |
| 37.40 | 1.1 | −0.003 | — | — | — | — |
| | | Global Fit: | <1.0E−12 | 1.190E+03 | <1.0E−07 | 0.667 |

K_{D} Affinity constant (M) = k_{dis}/kₒₙ
kₒₙ Binding rate (1/Ms)
k_{dis} Dissociation rate (1/s)
Full-R² Curve-fitting correlation coefficient

### Example 4. The Monoclonal Antibodies Did not Influence the Normal Growth of Lymphocyte

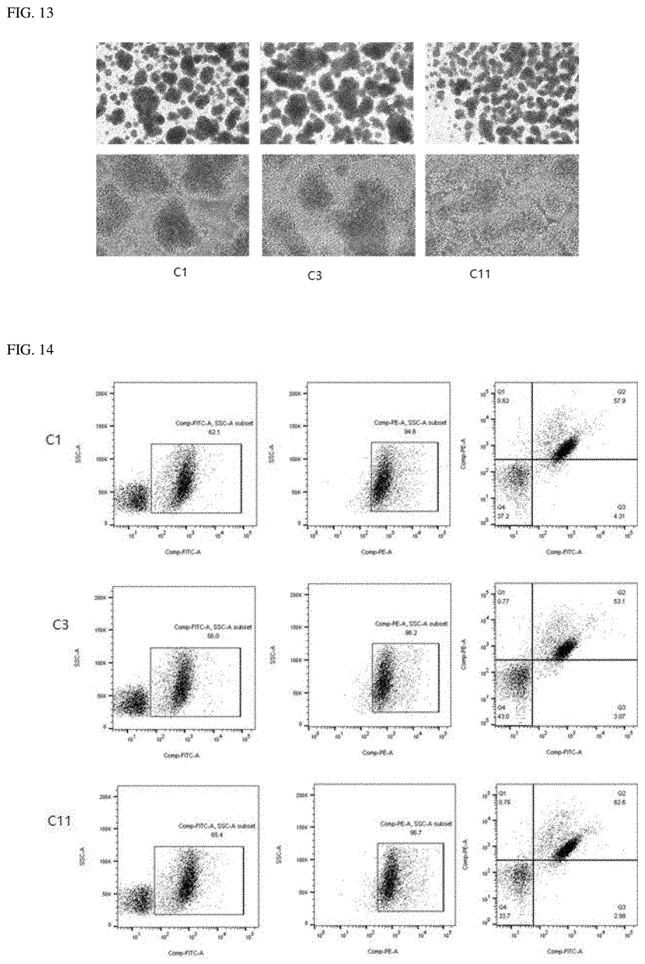

After being stimulated with 0.5 μg/mL ConA (concanavalin A), lymphocyte were amplified. The binding of caninized monoclonal antibodies C1, C3 and C11 to PBMC did not influence the amplification of lymphocyte. The upper and lower pictures in FIG. 13 correspond to the growth of PBMC observed under 4× and 10× lens, respectively. It can be seen that the cells are in a rapid growth period, and cell cloning is obvious. Therefore, the caninized monoclonal antibodies C1, C3 and C11 obtained by the present application do not influence the normal growth of lymphocyte.

### Example 5. The Monoclonal Antibodies can Effectively Bind to PD1 on the Surface of T Cells

The cells were stimulated by 0.5 μg/mL ConA for 3 days, and collected. PBMC was incubated with canine-anti-CD3-FITC and caninized monoclonal antibodies C1, C3 and C11 respectively. Then the cells were incubated again with PE-labeled canine-IgG antibody. The detection results show that T cells account for 62.1%, 56.0% and 65.4%, respectively, in total PBMC. PD1 positive cells account for 94.6%, 96.2% and 96.7%, respectively, in T cells, indicating that the caninized monoclonal antibodies C1, C3 and C11 can effectively bind to PD1 on the surface of T cells. Moreover, the results also indicate that the caninized monoclonal antibodies C1, C3 and C11 have potential blocking effect on the PD1/PD-L1 signaling pathway, and thus have protective effect on the function of T cells (see FIG. 14).

### Example 6. The Addition of the Monoclonal Antibodies Did not Influence the Release of Interferon

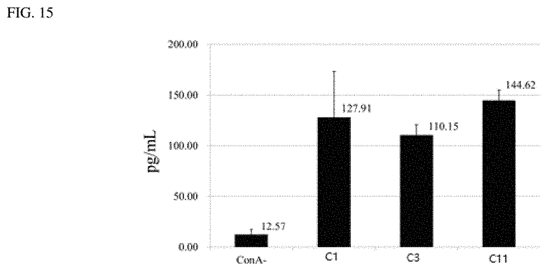

After being stimulated by 0.5 μg/mL ConA, lymphocyte were amplified. The amplified PBMC were taken, and incubated with the caninized monoclonal antibodies C1, C3 and C11 respectively to detect the influence of the addition of the antibodies on the release capacity of PBMC interferon. After the antibodies were bound to PBMC for 72 h, the supernatant was collected to detect the level of IFNγ. The results show that the interferon release levels of the activated PBMC are higher than that of the unactivated PBMC. Thus, the addition of the antibodies do not influence the release of interferon. In addition, there is no distinct difference among the three antibodies (see FIG. 15).

1-23. (canceled)

24. An anti-canine PD-1 antibody capable of binding with canine PD-1, comprising:
three light-chain complementary determining regions (CDR1-3) or a conservatively modified variant maintaining the function thereof; and/or
three heavy-chain complementary determining regions (CDR1-3) or a conservatively modified variant maintaining functions thereof.

25. The antibody according to claim 24, wherein
the light-chain complementary determining region CDR1 is selected from a group consisting of:

| (SEQ ID NO: 1) | |
|---|---|
| | TCAAGTGTAACTTAC, |
| (SEQ ID NO: 2) | |
| | TCAAGTGTAAGTTAC |
| | and |
| (SEQ ID NO: 3) | |
| | TCAAGTATAACTTAC; |

the light-chain complementary determining region CDR2 is selected from a group consisting of:

| (SEQ ID NO: 4) | |
|---|---|
| | GACACATCC; |

the light-chain complementary determining region CDR3 is selected from a group consisting of:
(SEQ ID NO: 5)
CAGCAGTACAGTGGTCACCCATCCTCG,
(SEQ ID NO: 6)
CAACAGTACAGTGGTTACCCGTACACG
and
(SEQ ID NO: 7)
CAGCAGTACAGTGGTTACCCATCCACG;
the heavy-chain complementary determining region CDR1 is selected from a group consisting of:
(SEQ ID NO: 8)
GGCTTCAACATCAAAGACACCTAT
and
(SEQ ID NO: 9)
GGCTTCAAC ATTAAAGACACCTAT;
the heavy-chain complementary determining region CDR2 is selected from a group consisting of:
(SEQ ID NO: 10)
ATTGATCCTGCGATTGATAATACT.
(SEQ ID NO: 11)
ATTGATCCTGCGA TTGGTAATACT
and
(SEQ ID NO: 12)
ATTGATCCTGCGATTGGTAATCCT;
the heavy-chain complementary determining region CDR3 is selected from a group consisting of:
(SEQ ID NO: 13)
GCTTCTGGGTTCTATACTATGGACTAC,
(SEQ ID NO: 14)
GCTAGAGGGTTCTATGGTATGGACTAC
and
(SEQ ID NO: 15)
GCTTCTGGGTTCTATGCTATGGACTGC.

26. The antibody according to claim 24, wherein the anti-canine PD-1 antibody comprises the following combination of the three light-chain complementary determining regions (CDR1-3):
(1) CDR1:
(SEQ ID NO: 1)
TCAAGTGTAACTTAC,
CDR2:
(SEQ ID NO: 4)
GACACATCC,
CDR3:
(SEQ ID NO: 5)
CAGCAGTACAGTGGTCACCCATCCTCG;
(2) CDR1:
(SEQ ID NO: 2)
TCAAGTGTAAGTTAC,
CDR2:
(SEQ ID NO: 4)
GACACATCC,
CDR3:
(SEQ ID NO: 6)
CAACAGTACAGTGGTTACCCGTACACG;
or
(3) CDR1:
(SEQ ID NO: 3)
TCAAGTATAACTTAC,
CDR2:
(SEQ ID NO: 4)
GACACATCC,
CDR3:
(SEQ ID NO: 7)
CAGCAGTACAGTGGTTACCCATCCACG.

27. The antibody according to claim 24, wherein the anti-canine PD-1 antibody comprises the following combination of the three heavy-chain complementary determining regions (CDR1-3):
(1) CDR1:
(SEQ ID NO: 8)
GGCTTCAACATCAAAGACACCTAT,
CDR2:
(SEQ ID NO: 10)
ATTGATCCTGCGATTGATAATACT,
CDR3:
(SEQ ID NO: 13)
GCTTCTGGGTTCTATACT ATGGACTAC;
(2) CDR1:
(SEQ ID NO: 9)
GGCTTCAACATTAAAGACACCTAT,
CDR2:
(SEQ ID NO: 11)
ATTGATCCTGCGATTGGTAATACT,
CDR3:
(SEQ ID NO: 13)
GCTTCTGGGTTCTATACTATGGACTAC;
or
(3) CDR1:
(SEQ ID NO: 9)
GGCTTCAACATTAAAGACACCTAT,
CDR2:
(SEQ ID NO: 12)
ATTGATCCTGCGATTGGTAATCCT,
CDR3:
(SEQ ID NO: 15)
GCTTCTGGGTTCTATGCTATGGACTGC.

28. The antibody according to claim 24, wherein the anti-canine PD-1 antibody comprises the following combination of the following three light-chain complementary determining regions (CDR1-3) and three heavy-chain complementary determining regions (CDR1-3):
(1) light chain CDR1: TCAAGTGTAACTTAC (SEQ ID NO: 1), light chain CDR2: GACACATCC(SEQ ID NO: 4), light chain CDR3: CAGCAGTACAGTGGTCACCCATCCTCG (SEQ ID NO: 5), heavy chain CDR1: GGCTTCAACATCAAAG ACACCTAT (SEQ ID NO: 8), heavy chain CDR2: ATTGATCCTGCGATTGATA ATACT (SEQ ID NO: 10) and heavy chain CDR3: GCTTCTGGGTTCTATACTATGG ACTAC (SEQ ID NO: 13);
(2) light chain CDR1: TCAAGTGTAAGTTAC (SEQ ID NO: 2), light chain CDR2: GACACATCC(SEQ ID NO: 4), light chain CDR3: CAACAGTACAGTGGTTACCCGTACACG (SEQ ID NO: 6), heavy chain CDR1: GGCTTCAACATTAAAGACACCTAT (SEQ ID NO: 9), heavy chain CDR2: ATTGA TCCTGCGATTGGTAATACT (SEQ ID NO: 11), heavy chain CDR3: GCTTCTGGGTT CTATACTATGGACTAC (SEQ ID NO: 13); or
(3) light chain CDR1: TCAAGTATAA CTTAC(SEQ ID NO: 3), light chain CDR2: GACACATCC (SEQ ID NO: 4), light chain CDR3: CAGCAGTACAGTGGTTACCCATCCACG (SEQ ID NO: 7), heavy chain CDR1: GGCTTCAACATTAAAGACACCTAT (SEQ ID NO: 9), heavy chain CDR2: ATTG ATCCTGCGATTGGTAATCCT (SEQ ID NO: 12) and heavy chain CDR3: GCTTCTGGG TTCTATGCTATGGACTGC (SEQ ID NO: 15).

29. The antibody according to claim 24, wherein the anti-canine PD-1 antibody comprises a light chain variable region selected from a group consisting of:
(1)
(SEQ ID NO: 16)
GCAAATTGTTCTCACCCAGTCTCCAGCAATCATGT
CTGCATCTCCAGGGGAGAAGGTCACCATGACCTGC
AGTGCCAGCTCAAGTGTAACTTACATGTATTGGTT
CCAGCAGAAGCCAGGCTCCTCCCCCAGACTCTGGA
TTTATGACACATCCAACCTGGTTTCTGGAGTCCCT
GCTCGCTTCAGTGGCAGTAGGTCTGGGACCTCTTA
TTCTCTCACAATCAGCATATGGAGGCTGAAGATGC
TGCCACTTATTACTGCCAGCAGTACAGTGGTCACC
CATCCTCGTTCGGCTCGGGGACAAAGTTGGAAATT
AAA;
(2)
(SEQ ID NO: 17)
CAAATTGTTCTCACCCAGTCTCCAGCAATCATGTC
TGCATCTCCAGGGGAGAAGGTCACCATGACCTGCA
GTGCCAGCTCAAGTGTAAGTTACATGTTCTGGTAC
CAGCAGAAGCCAGGCTCCTCCCCCAGACTCTGGAT
TTATGACACATCCAACCTGGTTTCTGGAGTCCCTG
CTCGCTTCAGTGGCAGTAGGTCTGGGACCTCTTAT
TCTCTCACAATCAGCAGCATGGAGGCTGAAGATGC
TGCCACTTATTACTGCCAACAGTACAGTGGTTACC
CGTACACGTTCGGAGGGGGGACCAAGCTGGAAATA
AAG;
and
(3)
(SEQ ID NO: 18)
CAAATTGTTCTCACCCAGTCTCCAGCCATCATGTC
TGCATCTCCAGGGGAAAAGGTCACCATGACCTGCA
GTGCCAGCTCAAGTATAACTTACATGTTCTGGTAC
CAGCAGAAGCCAGGCTCCTCCCCCAGACTCTGGAT
TTATGACACATCCAACCTGGTTTCTGGAGTCCCTG
CTCGCTTCAGTGGCAGTAAGTCTGGGACCTCTTAT
TCTCTCACAATCACCAGCATGGAGGCTGAAGATGC
TGCCACTTATTACTGCCAGCAGTACAGTGGTTACC
CATCCACGTTCGGCTCGGGGACAAAGTTGGAAATA
AAA.

30. The antibody according to claim 24, wherein the anti-canine PD-1 antibody comprises a heavy chain variable region nucleotide sequence selected from a group consisting of:
(1)
(SEQ ID NO: 19)
GAGGTTCAGCTGCAGCAGTCTGGGGCAGAGCTTGT
GAAGCCAGGGGCCTCAGTCAAGTTGTCCTGCACAG
CTTCTGGCTTCAACATCAAAGACACCTATATGCAT
TGGGTGAAGCAGAGGCCTGAACAGGGCCTGGAGTG
GATTGGAAGGATTGATCCTGCGATTGATAATACTA
AATATGACCCGAAGTTCCAGGGCAAGGCCACTATA
ACAGCTGACACATCCTCCAACACAGCCTACCTGCA
GCTCAGCAGCCTGACATCTGAGGACACTGCCGTCT
ATTACTGTGCTTCTGGGTTCTATACTATGGACTAC
TGGGGTCAAGGAACCTCAGTCACCGTCTCCTCA;
(2)
(SEQ ID NO: 20)
GAGGTTCGGCT
GCAGCAGTCTGGGGCAGAGCTTGTGAAGCCAGGGG
CCTCAGTCAAGTTGTCCTGCACAGCTTCTGGCTTC
AACATTAAAGACACCTATTTACACTGGTTGAAGGA
GAGGCCTGAACAGGGCCTGGAGTGGATTGGAAGGA
TTGATCCTGCGATTGGTAATACTAGATATGACCCG
AAGTTCCAGGTCAAGGCCACTATAACAGCAGACAC
ATCCTCCAACACAGCCTACCTGCAACTCAGCAGCC
TGACATCTGAGGACTCTGCCGTCTATTACTGTGCT
AGAGGGTTCTATGGTATGGACTACTGGGGTCAAGG
AACCTCAGTCACCGTCTCCTCA;
and
(3)
(SEQ ID NO: 21)
GAGGTTCAGCTGCAGCAGTCTGGGGCAGAGGTTGT
GAAGCCAGGGGCCTCAGTCAAGTTGTCCTGCACAG
CTTCTGGCTTCAACATTAAAGACACCTATATGCAC
TGGGTGAAGCAGAGGCCTGAACAGGGCCTGGAGTG
GATTGGAAGGATTGATCCTGCGATTGGTAATCCTA
AATATGACCCGAAGTTCCAGGGCAGGGCCACTATA
ACTGCTGACACATCCTCCAACACAGCCTACCTGCA
GCTCAGCAGCCTGACATCTGAGGACACTGCCGTCT
ATTACTGTGCTTCTGGGTTCTATGCTATGGACTGC
TGGGGTCAAGGAACCTCAGTCACCGTCTCCTCA.

31. The antibody according to claim 24, wherein the anti-canine PD-1 antibody comprises the following combination of the light chain variable regions and heavy chain variable regions:
(1) a light chain variable region as shown by SEQ ID NO: 16 and a heavy chain variable region as shown by SEQ ID NO: 19;
(2) a light chain variable region as shown by SEQ ID NO: 17 and a heavy chain variable region as shown by SEQ ID NO: 20; or
(3) a light chain variable region as shown by SEQ ID NO: 18 and a heavy chain variable region as shown by SEQ ID NO: 21.

32. The antibody according to claim 24, wherein the anti-canine PD-1 antibody is a mouse-anti-canine PD-1 antibody, preferably, the anti-canine PD-1 antibody is a mouse-anti-canine PD-1 monoclonal antibody, and the anti-canine PD-1 antibody blocks the binding of canine PD-1 to canine PD-L1.

33. The antibody according to claim 24, wherein the anti-canine PD-1 antibody has an affinity of 1.000 E⁰⁷-1.000 E¹².

34. The antibody according to claim 24, wherein the anti-canine PD-1 antibody is a caninized anti-canine PD-1 monoclonal antibody, and the caninized anti-canine PD-1 monoclonal antibody block the binding of the canine PD-1 to canine PD-L1.

35. The antibody according to claim 34, wherein the caninized anti-canine PD-1 monoclonal antibody comprises a heavy chain constant region sequence and/or a light chain constant region sequence of the canine PD-1 monoclonal antibody; preferably, the heavy chain constant region has a sequence as shown by SEQ ID NO: 28, and the light chain constant region has a sequence as shown by SEQ ID NO: 29.

36. A functional fragment of an anti-canine PD-1 antibody capable of binding with canine PD-1, comprising three light-chain complementary determining regions (CDR1-3) or a combination of conservatively modified variants maintaining the function thereof, or comprising three heavy-chain complementary determining regions (CDR1-3) or a combination of conservatively modified variants maintaining functions thereof;
the light-chain complementary determining region CDR1 is selected from a group consisting of:
(SEQ ID NO: 1)
TCAAGTGTAACTTAC
(SEQ ID NO: 2)
TCAAGTGTAAGTTAC
and
(SEQ ID NO: 3)
TCAAGTATAACTTAC;
the light-chain complementary determining region CDR2 is: GACACATCC (SEQ ID NO: 4);
the light-chain complementary determining region CDR3 is selected from a group consisting of:
(SEQ ID NO: 5)
CAGCAGTACAGTGGTCACCCATCCTCG,
(SEQ ID NO: 6)
CAACAGTACAGTGGTTACCCGTACACG
and
(SEQ ID NO: 7)
CAGCAGTACAGTGGTTACCCATCCACG;
the heavy-chain complementary determining region CDR1 is selected from a group consisting of:
(SEQ ID NO: 8)
GGCTTCAACATCAAAGACACCTAT
and
(SEQ ID NO: 9)
GGCTTCAACATTAAAGACACCTAT;
the heavy-chain complementary determining region CDR2 is selected from a group consisting of:
(SEQ ID NO: 10)
ATTGATCCTGCGATTGATAATACT,
(SEQ ID NO: 11)
ATTGATCCTGCGATTGGTAATACT
and
(SEQ ID NO: 12)
ATTGATCCTGCGATTGGTAATCCT;
the heavy-chain complementary determining region CDR3 is selected from a group consisting of:
(SEQ ID NO: 13)
GCTTCTGGGTTCTATACTATGGACTAC,
(SEQ ID NO: 14)
GCTAGAGGGTTCTATGGTATGGACTAC
and
(SEQ ID NO: 15)
GCTTCTGGGTTCTATGCTATGGACTGC.

37. The fragment according to claim 36, wherein the functional fragment of the anti-canine PD-1 antibody comprises the following combination of the three light-chain complementary determining regions (CDR1-3):
(1)
CDR1:
(SEQ ID NO: 1)
TCAAGTGTAACTTAC,
CDR2:
(SEQ ID NO: 4)
GACACATCC,
CDR3:
(SEQ ID NO: 5)
CAGCAGTACAGTGGTC ACCCATCCTCG;
(2)
CDR1:
(SEQ ID NO: 2)
TCAAGTGTAAGTTAC,
CDR2:
(SEQ ID NO: 4)
GACACATCC,
CDR3:
(SEQ ID NO: 6)
CAACAGTACAGT GGTTACCCGTACACG;
or
(3)
CDR1:
(SEQ ID NO: 3)
TCAAGTATAAC TTAC,
CDR2:
(SEQ ID NO: 4)
GACACATCC,
CDR3:
(SEQ ID NO: 7)
CAGCAGTACAGTGGTTACCCATCCACG
or a combination of the conservatively modified variants maintaining functions thereof;
and/or, wherein the functional fragment of the anti-canine PD-1 antibody comprises the following combination of the three heavy-chain complementary determining regions (CDR1-3):
(1) CDR1:
(SEQ ID NO: 8)
GGCTTCAACATCAAAGACACCTAT,
CDR2:
(SEQ ID NO: 10)
ATTGATCCTGCGATTGATAATACT,
CDR3:
(SEQ ID NO: 13)
GCTTCTGGGTTCTATACTATGGACTAC;
(2) CDR1:
(SEQ ID NO: 9)
GGCTTCAACATTAAAGACACCTAT,
CDR2:
(SEQ ID NO: 11)
ATTGATCCTGCGATTGGTAATACT,
CDR3:
(SEQ ID NO: 13)
GCTTCTGGGTTCTATACTATGGACTAC;
or
(3) CDR1:
(SEQ ID NO: 9)
GGCTT CAACATTAAAGACACCTAT;
CDR2:
(SEQ ID NO: 12)
ATTGATCCTGCGATTGGTAATCCT;
CDR3:
(SEQ ID NO: 15)
GCTTCTGGGTTCTATGCTATGGACTGC,
or a combination of the conservatively modified variants maintaining functions thereof.

38. The fragment according to claim 36, wherein the functional fragment of the anti-canine PD-1 antibody comprises the following combination of the three light-chain complementary determining regions (CDR1-3) and three heavy-chain complementary determining regions (CDR1-3):
(1) light chain CDR1: TCAAGTGTAACTTAC (SEQ ID NO: 1), light chain CDR2: GACACATCC (SEQ ID NO: 4), light chain CDR3: CAGCAGTACAGTGGTCACCCATCCTCG (SEQ ID NO: 5), heavy chain CDR1: GGCTTCAACATCAAAGACACCTAT (SEQ ID NO: 8), heavy chain CDR2: ATTGATCCTGCGATTGATAATACT (SEQ ID NO: 10), heavy chain CDR3: GCTTCTGGGTTCTATACTATGGACTAC (SEQ ID NO: 13);
(2) light chain CDR1: TCAAGTGTAAGTTAC (SEQ ID NO: 2), light chain CDR2: GACACATCC (SEQ ID NO: 4), light chain CDR3: CAACAGTACAGTGGTTACCC GTACACG (SEQ ID NO: 6), heavy chain CDR1: GGCTTCAACATTAAAGACACC TAT (SEQ ID NO: 9), heavy chain CDR2: ATTGATCCTGCGATTGGTAATACT (SEQ ID NO: 11), heavy chain CDR3: GCTTCTGGGTTCTATACTATGGACTAC (SEQ ID NO: 13); or
(3) light chain CDR1: TCAAGTATAACTTAC (SEQ ID NO: 3), light chain CDR2: GACACATCC (SEQ ID NO: 4), light chain CDR3: CAGCAGTACAGTGGTTACCCATCCACG (SEQ ID NO: 7), heavy chain CDR1: GGCTTCAACATTAA AGACACCTAT (SEQ ID NO: 9), heavy chain CDR2: ATTGATCCTGCGATTGGTA ATCCT (SEQ ID NO: 12), heavy chain CDR3: GCTTCTGGGTTCTATGCTATGGAC TGC (SEQ ID NO: 15),
or a combination of the conservatively modified variants maintaining functions thereof.

39. The fragment according to claim 36, wherein the functional fragment of the anti-canine PD-1 antibody comprises the following combination of the three light-chain complementary determining regions (CDR1-3) and three heavy-chain complementary determining regions (CDR1-3):
(1) light chain CDR1: TCAAGTGTAACTTAC (SEQ ID NO: 1), light chain CDR2: GACACATCC (SEQ ID NO: 4), light chain CDR3: CAGCAGTACAGTGGTCACCCATCCTCG (SEQ ID NO: 5), heavy chain CDR1: GGCTTCAACATCAAAGACACCTAT (SEQ ID NO: 8), heavy chain CDR2: ATTGATCCTGCGATT GATAATACT (SEQ ID NO: 10), heavy chain CDR3: GCTTCTGGGTTCTATACTAT GGACTAC (SEQ ID NO: 13);
(2) light chain CDR1: TCAAGTGTAAGTTAC (SEQ ID NO: 2), light chain CDR2: GACACATCC (SEQ ID NO: 4), light chain CDR3: CAACAGTACAGTGGTTACCCGTACACG (SEQ ID NO: 6), heavy chain CDR1: GGCTTCAACATTAAAGACACCTAT (SEQ ID NO: 9), heavy chain CDR2: ATTGA TCCTGCGATTGGTAATACT (SEQ ID NO: 11), heavy chain CDR3: GCTTCTGGGT TCTATACTATGGACTAC (SEQ ID NO: 13); or
(3) light chain CDR1: TCAAGTATA ACTTAC (SEQ ID NO: 3), light chain CDR2: GACACATCC (SEQ ID NO: 4), light chain CDR3: CAGCAGTACAGTGGTTACCCATCCACG (SEQ ID NO: 7), heavy chain CDR1: GGCTTCAACATTAAAGACACCTAT (SEQ ID NO: 9), heavy chain CDR2: ATTGATCCTGCGATTGGTAATCCT (SEQ ID NO: 12), heavy chain CDR3: GCTTCTGGGTTCTATGCTATGGACTGC (SEQ ID NO: 15).

40. The fragment according to claim 36, wherein the functional fragment of the anti-canine PD-1 antibody comprises a light chain variable region nucleotide sequence selected from a group consisting of:
(1) a nucleotide sequence as shown by SEQ ID NO: 16;
(2) a nucleotide sequence as shown by SEQ ID NO: 17; and
(3) a nucleotide sequence as shown by SEQ ID NO: 18;
and/or, wherein the functional fragment of the anti-canine PD-1 antibody comprises a heavy chain variable region nucleotide sequence selected from a group consisting of:
(1) a nucleotide sequence as shown by SEQ ID NO: 19;
(2) a nucleotide sequence as shown by SEQ ID NO: 20; and
(3) a nucleotide sequence as shown by SEQ ID NO: 21.

41. The fragment according to claim 36, wherein the functional fragment of the anti-canine PD-1 antibody comprises the following combination of the light chain variable regions and heavy chain variable regions:
(1) a light chain variable region as shown by SEQ ID NO: 16 and a heavy chain variable region as shown by SEQ ID NO: 19;
(2) a light chain variable region as shown by SEQ ID NO: 17 and a heavy chain variable region as shown by SEQ ID NO: 20; or
(3) a light chain variable region as shown by SEQ ID NO: 18 and a heavy chain variable region as shown by SEQ ID NO: 21,
or a combination of the conservatively modified variants maintaining functions thereof.

42. The fragment according to claim 36, wherein the functional fragment is an antigen-binding fragment.

43. A method for treating canine cancers by using the antibody according to claim 24.
